SEQUENCE LISTING (1 sequence)

SEQ ID NO: 1 - DNA, 101 bp, synthetic construct
  source 1..101: mol_type = other DNA
ttaccgcggckgctgrcacaaaaaaaaaaaaaaaaaaaaaaaaaaaaaannnngaagagcccttggcacccgagaattccaggactacnvgggtwtctaat

(19) United States
(12) Patent Application Publication
(10) Pub. No.: US 2025/0340933 A1
(43) Pub. Date: Nov. 6, 2025

(54) METHODS, COMPOSITIONS, AND KITS FOR DETERMINING THE PRESENCE AND/OR LOCATION OF AN EXOGENOUS TARGET NUCLEIC ACID IN A BIOLOGICAL SAMPLE

(71) Applicant: 10x Genomics, Inc., Pleasanton, CA (US)

(72) Inventors: Stefania Giacomello, Stockholm (SE); Hailey Elizabeth Sounart, Stockholm (SE)

(21) Appl. No.: 19/196,392

(22) Filed: May 1, 2025

Related U.S. Application Data
(60) Provisional application No. 63/641,140, filed on May 1, 2024.

(51) Int. Cl. C12Q 1/6841

(52) U.S. Cl. CPC C12Q 1/6841

(57) ABSTRACT

Provided herein are methods, compositions, and kits for determining the spatial location of target nucleic acids, including endogenous and exogenous target nucleic acids, in a biological sample using padlock probes and substrates with spatially barcoded capture probes. Also disclosed herein are methods for determining a presence and/or location of a microbe (e.g., archaea, fungi, bacteria) in a biological sample, e.g., by determining the presence and/or location of a microbial target nucleic acid in the biological sample.

### CROSS-REFERENCE TO RELATED APPLICATIONS

This application claims the benefit of priority to U.S. Provisional Patent Application No. 63/641,140, filed on May 1, 2024, the contents of which are herein incorporated by reference in their entirety.

### SEQUENCE LISTING

This application contains a Sequence Listing that has been submitted electronically as an XML file named 47706-0407001_SL_ST26.xml. The XML file, created on Apr. 25, 2025, is 2,075 bytes in size. The material in the XML file is hereby incorporated by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small handful of analytes in the context of an intact tissue or a portion of a tissue or provides substantial analyte data for dissociated tissue (i.e., single cells), but fail to provide information regarding the position of the single cell in a parent biological sample (e.g., tissue sample).

While advances in spatial transcriptomics have improved understanding of gene expression mechanisms, e.g., in relation to development and disease, spatially resolved analyses of host-microbial interactions has been limited. Typically, spatial analysis utilizes capture probes with capture domains capable of targeting particular analytes in a biological sample with poly(A) sequences (e.g., mRNA). However, such capture probes do not capture non-polyadenylated nucleic acids including, for example, exogenous target nucleic acids from a microbe present in a biological sample. Thus, there remains a need to develop spatial analysis methods that include capture of both endogenous (e.g., host) target nucleic acids and exogenous (e.g., microbial) target nucleic acids within a biological sample.

### SUMMARY

Disclosed herein are compositions, kits and methods to facilitate an increased understanding of the spatial organization of microorganisms within hosts, and the associated local host responses. The present disclosure features methods, compositions, and kits for determining the location of endogenous and/or exogenous target nucleic acids in a biological sample. The methods described herein can be used across various types of biological samples to identify exogenous (e.g., archaeal, bacterial, fungal, etc.) nucleic acids, and by extension, microbes present in a biological sample (e.g., a plant sample, a mammalian sample, etc.). Understanding pathology of exogenous organisms, including for example, location of bacterial, viral, and/or fungal exogenous nucleic acids within a biological sample helps elucidate molecular mechanisms of infection and potential treatments and/or therapies.

Thus, described herein are methods for determining the presence and/or location of a microbe (e.g., archaea, bacteria, fungi) in a biological sample. In particular, described herein are methods for determining the presence and/or location of endogenous and/or exogenous target nucleic acids in a biological sample. More specifically, in some embodiments, exogenous target nucleic acids are detected with padlock probes that can be subsequently captured on a substrate including a plurality of spatially barcoded capture probes and further processed to determine the presence and/or location of the exogenous target nucleic acid as detailed more fully herein.

Thus provided herein are methods for determining a location of a target nucleic acid in a biological sample including: a) contacting the biological sample with a plurality of padlock probes, where a padlock probe of the plurality of padlock probes includes a first sequence substantially complementary to the target nucleic acid, a capture probe binding domain, a cleavage site, and a second sequence substantially complementary to the target nucleic acid; b) hybridizing the padlock probe to the target nucleic acid; c) extending the second sequence substantially complementary to the target nucleic acid, thereby generating an extended padlock probe; d) ligating a first end of the extended padlock probe to a second end of the extended padlock probe, thereby generating a ligated padlock probe; e) cleaving the cleavage site of the ligated padlock probe, thereby generating a linear padlock probe; f) hybridizing the capture probe binding domain of the linear padlock probe to the capture domain of a capture probe on a first substrate, where the capture probe is included in a plurality of capture probes on the first substrate, the capture probe including: i) a spatial barcode and ii) the capture domain; and g) determining the sequence of (i) the spatial barcode, or a complement thereof, and (ii) all or a part of the sequence of the linear padlock probe, or a complement thereof; and using the sequences of (i) and (ii) to determine the location of the target nucleic acid in the biological sample.

In some embodiments, the biological sample is disposed on the first substrate including the plurality of capture probes. In some embodiments, the biological sample is disposed on a second substrate. In some embodiments, the method includes aligning the second substrate including the biological sample with the first substrate, such that at least a portion of the biological sample is aligned with at least a portion of the first substrate.

In some embodiments, the capture probe includes a unique molecular identifier, a cleavage domain, a sequencing specific site, and/or a primer binding site.

In some embodiments, the first sequence substantially complementary to the target nucleic acid and the second sequence substantially complementary to the target nucleic acid hybridize to conserved regions of the target nucleic acid. In some embodiments, the first sequence substantially complementary to the target nucleic acid and the second sequence substantially complementary to the target nucleic acid flank a variable region of the target nucleic acid.

In some embodiments, the padlock probe includes in a 5′ to 3′ direction: (i) the first sequence substantially complementary to the target nucleic acid; (ii) the capture probe binding domain; (iii) the cleavage site; and (iv) the second sequence substantially complementary to the target nucleic acid.

In some embodiments, the padlock probe includes a functional domain, optionally a sequencing specific site or a primer binding site.

In some embodiments, the method includes releasing the ligated padlock probe from the target nucleic acid, optionally where the releasing is performed prior to step (f), where the releasing includes use of one or more RNases.

In some embodiments, the ligating is performed using a ligase selected from the group consisting of: Tth DNA ligase, Taq DNA ligase, Thermococcus sp. DNA ligase, PBCV-1 DNA Ligase, and Chlorella virus DNA Ligase.

In some embodiments, the method includes extending the capture probe using the linear padlock probe as a template, thereby generating an extended capture probe, and/or extending the linear padlock probe using the capture probe as a template, thereby generating an extended linear padlock probe.

In some embodiments, the determining step includes sequencing the extended capture probe or a complement thereof, or the extended linear padlock probe or a complement thereof.

In some embodiments, the biological sample is derived from a mammal or a plant, optionally where the target nucleic acid is exogenous to the biological sample.

In some embodiments, the target nucleic acid includes archaeal RNA, bacterial RNA, fungal RNA, or a combination thereof. In some embodiments, the bacterial RNA is bacterial ribosomal RNA including 16S ribosomal RNA or 5S ribosomal RNA, or where the fungal RNA is fungal RNA including 18S ribosomal RNA or internal transcribed spacer (ITS) region ribosomal RNA.

In some embodiments, the biological sample is a tissue section, optionally a fixed tissue section or a fresh-frozen tissue section.

Also provided herein are kits including: a) a substrate including a plurality of capture probes, where a capture probe of the plurality of capture probes includes: i) a spatial barcode and ii) a capture domain; b) a plurality of padlock probes, where a padlock probe of the plurality of padlock probes includes a first sequence substantially complementary to a target nucleic acid, a capture probe binding domain, a cleavage site, a functional domain, and a second sequence substantially complementary to the target nucleic acid; and c) one or more enzymes.

Also provided herein are compositions including: a target nucleic acid, and a plurality of ligated padlock probes, where a ligated padlock probe of the plurality of ligated padlock probes includes a first sequence substantially complementary to a target nucleic acid, a capture probe binding domain complementary to a capture domain of a capture probe on a substrate, a cleavage site, and a second sequence substantially complementary to the target nucleic acid, where the ends of the ligated padlock probe are ligated to each other, and where the ligated padlock probe is hybridized to the target nucleic acid.

Also provided herein are methods for determining a presence and/or location of a microbial target nucleic acid in a biological sample including: a) providing a first substrate including a plurality of capture probes, where a capture probe of the plurality of capture probes includes: i) a spatial barcode and ii) a capture domain; b) contacting the biological sample with a plurality of padlock probes, where a padlock probe of the plurality of padlock probes includes a first sequence substantially complementary to the microbial target nucleic acid, a capture probe binding domain, a cleavage site, and a second sequence substantially complementary to the microbial target nucleic acid; c) hybridizing the padlock probe to the microbial target nucleic acid; d) ligating ends of the extended padlock probe to each other, thereby generating a ligated padlock probe; e) cleaving the cleavage site of the ligated padlock probe, thereby generating a linear padlock probe; f) hybridizing the capture probe binding domain of the linear padlock probe to the capture domain of the capture probe on the first substrate; and g) determining the sequence of (i) the spatial barcode and (ii) all or a part of the sequence of the linear padlock probe; and using the sequences of (i) and (ii) to determine the presence and/or location of the microbial target nucleic acid in the biological sample; optionally where the microbial target nucleic acid is derived from a microbe selected from the group including fungi, bacteria, archaea, or a combination thereof, optionally where the microbe is a pathogenic microbe.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “substantially complementary” used herein means that a first sequence is at least 50%, 55%, 60%, 65%, 70%, 75%, 80%, 85%, 90%, 95%, 97%, 98% or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, or that the two sequences hybridize under stringent hybridization conditions. Substantially complementary also means that a sequence in one strand is not necessarily completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations known to those skilled in the art.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows an exemplary workflow for performing templated capture and producing a ligation product, and FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe 1124 and an analyte capture agent 1126.

FIG. 12 is a schematic diagram showing an exemplary workflow for detection of a target nucleic acid with a padlock probe and capture of a linearized padlock probe via a capture probe on a substrate including spatially barcoded capture probes.

### DETAILED DESCRIPTION

### A. Spatial Analysis Methods

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; and the Visium Spatial Gene Expression Reagent Kits User Guide (e.g., Rev F, dated January 2022) and/or the Visium Spatial Gene Expression Reagent Kits—Tissue Optimization User Guide (e.g., Rev E, dated February 2022), both of which are available at the 10× Genomics Support Documentation website, and can be used herein in any combination, and each of which is incorporated herein by reference in its entirety. Further non-limiting aspects of spatial analysis methodologies and compositions are described herein.

Some general terminology that may be used in this disclosure can be found in Section (I)(b) of PCT Publication No. WO2020/176788 and/or U.S. Patent Application Publication No. 2020/0277663, which is herein incorporated by reference. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Publication No. WO2020/176788 and/or U.S. Patent Application Publication No. 2020/0277663, which is herein incorporated by reference. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples—which can be from different tissues or organisms—assembled on a single histologic slide. The TMA can therefore allow for high throughput analysis of multiple specimens at the same time. Tissue microarrays may be paraffin blocks produced by extracting cylindrical tissue cores from different paraffin donor blocks and re-embedding these tissue cores into a single recipient (microarray) block at defined array coordinates.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section (e.g., a fixed tissue section). In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plant, an insect, an arachnid, a nematode (e.g., Caenorhabditis elegans), a fungus, an amphibian, or a fish (e.g., zebrafish)). A biological sample can be obtained from a prokaryote such as a bacterium, e.g., Escherichia coli, Staphylococci or Mycoplasma pneumoniae; an archaeon; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example, methanol. In some embodiments, instead of methanol, acetone or an acetone-methanol mixture can be used. In some embodiments, the fixation is performed after sectioning. In some instances, when the biological sample is fixed using a fixative including an alcohol (e.g., methanol or acetone-methanol mixture), the biological sample is not decrosslinked afterward. In some preferred embodiments, the biological sample is fixed using a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample, e.g., using acetone and/or alcohol (e.g., methanol, acetone-methanol), is performed while the sample is mounted on a substrate (e.g., glass slide, such as a positively charged glass slide).

In some embodiments, a substrate of the present technology includes a surface comprising one or more spatially barcoded capture probes, wherein the spatial barcodes are present at known spatial locations on the substrate. In some embodiments, a substrate of the present technology includes a surface comprising one or more spatially barcoded capture probes that are arranged in an ordered manner, such as a grid. In some embodiments, a substrate of the present technology includes a surface comprising one or more spatially barcoded capture probes, wherein the spatially barcoded capture probes are provided in a known but non-ordered manner, such as a random or irregular manner.

In some embodiments, a substrate of the present technology comprises an array (such as an ordered or non-ordered array). In some embodiments, a substrate of the present technology comprises an array of spatially barcoded capture probes present on the substrate surface in an ordered manner, such as a grid. In some embodiments, a substrate of the present technology includes a surface comprising an array of spatially barcoded capture probes present on the substrate surface in a non-ordered manner, such as a random or irregular manner.

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing, e.g., by formalin or PFA, the biological sample is dehydrated via sucrose gradient. In some instances, the fixed biological sample is treated with a sucrose gradient and then embedded in a matrix, e.g., OCT compound. In some instances, the fixed biological sample is not treated with a sucrose gradient, but rather is embedded in a matrix, e.g., OCT compound after fixation. In some embodiments when a fixed frozen tissue sample is treated with a sucrose gradient, the sample can be rehydrated using an ethanol gradient. In some embodiments, the PFA or formalin fixed biological sample, which can be optionally dehydrated via sucrose gradient and/or embedded in OCT compound, is then frozen, e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated using an ethanol gradient.

In some instances, the biological sample (e.g., a fixed frozen tissue sample) is treated with a citrate buffer. Citrate buffer can be used to decrosslink antigens and fixation medium for antigen retrieval in the biological sample. Thus, any suitable decrosslinking agent can be used in addition, or alternatively, to citrate buffer. In some embodiments, for example, the biological sample (e.g., a fixed frozen tissue sample) is decrosslinked using TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, the sample is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, the sample can be rehydrated using an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, an acid, and a soluble organic compound that preserves morphology and biomolecules. PAXgene provides a two-reagent fixative system in which tissue is firstly fixed in a solution containing methanol and acetic acid, then stabilized in a solution containing ethanol. See, Ergin B. et al., J Proteome Res. 2010 Oct. 1; 9(10):5188-96; Kap M. et al., PLoS One.; 6(11):e27704 (2011); and Mathieson W. et al., Am J Clin Pathol.; 146(1):25-40 (2016), each of which is hereby incorporated by reference in its entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample, is fixed, for example in methanol, acetone, acetone-methanol, PFA, PAXgene, or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RNA-templated ligation (RTL) methods disclosed herein. A limitation of direct RNA capture for fixed samples is that the RNA integrity of fixed (e.g., FFPE) samples can be lower than of a fresh sample, thereby capturing RNA directly from fixed samples, e.g., by capture of a common sequence such as a poly(A) tail of an mRNA molecule, can be more difficult. By utilizing RTL probes that hybridize to RNA target sequences in the transcriptome, RNA analytes can be captured without requiring that both a poly(A) tail and target sequences remain intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Biological samples are also described in Section (I)(d) of PCT Publication No. WO2020/176788 and/or U.S. Patent Application Publication No. 2020/0277663, which is herein incorporated by reference.

The following embodiments can be used with any of the methods described herein. In some embodiments, the biological sample (e.g., a fixed and/or stained biological sample) is imaged. In some embodiments, the biological sample is visualized or imaged using bright field microscopy. In some embodiments, the biological sample is visualized or imaged using fluorescence microscopy. The biological sample can be visualized or imaged using additional methods of visualization and imaging known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for analyzing captured analytes, as disclosed herein, to the biological sample.

In some embodiments, the methods include staining the biological sample. In some embodiments, the staining includes the use of hematoxylin and/or eosin. Non-limiting examples of stains include histological stains (e.g., hematoxylin and/or eosin) and immunological stains (e.g., fluorescent stains). In some embodiments, a biological sample can be stained using any number of biological stains, including but not limited to, acridine orange, Bismarck brown, carmine, coomassie blue, cresyl violet, DAPI (4′, 6-diamidino-2-phenylindole), eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label, such as a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Publication No. WO2020/176788 and/or U.S. Patent Application Publication No. 2020/0277663, which is herein incorporated by reference. Briefly, any of the methods described herein includes permeabilizing the biological sample. For example, the biological sample can be permeabilized to facilitate transfer of extension products to the capture probes on the substrate. In some embodiments, the permeabilizing includes the use of an organic solvent (e.g., acetone, ethanol, or methanol), a detergent (e.g., saponin, Triton X-100™, Tween-20™, or sodium dodecyl sulfate (SDS)), an enzyme (e.g., an endopeptidase, an exopeptidase, or a protease), or a combination thereof. In some embodiments, the permeabilizing includes the use of an endopeptidase, a protease, SDS, polyethylene glycol tert-octylphenyl ether, polysorbate 80, polysorbate 20, N-lauroylsarcosine sodium salt solution, saponin, Triton X-100™, Tween-20™, or a combination thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, which is herein incorporated by reference.

Spatial analysis methods can involve the transfer of one or more analytes or derivatives thereof from a biological sample to a substrate of features on a surface, such as a slide, where each feature is associated with a unique spatial location on the substrate. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the substrate, and the feature's relative spatial location within the substrate.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Publication No. WO2020/176788 and/or U.S. Patent Application Publication No. 2020/0277663, which is herein incorporated by reference. Generation of capture probes can be achieved by any appropriate method, including those described in Section (II)(d)(ii) of PCT Publication No. WO2020/176788 and/or U.S. Patent Application Publication No. 2020/0277663, which is herein incorporated by reference.

In some instances, a capture probe and a nucleic acid analyte interaction (or any other nucleic acid to nucleic acid interaction) occurs because the sequences of the two nucleic acids are substantially complementary to one another. By “substantial,” “substantially,” and the like, two nucleic acid sequences can be complementary when at least 60% of the nucleotide residues of one nucleic acid sequence are complementary to nucleotide residues of the other nucleic acid sequence. The complementary residues within a particular complementary nucleic acid sequence need not always be contiguous with each other, but can be interrupted by one or more non-complementary residues within the complementary nucleic acid sequence. In some embodiments, at least 60%, but less than 100%, of the residues of one of the two complementary nucleic acid sequences are complementary to residues of the other nucleic acid sequence. In some embodiments, at least 70%, 80%, 90%, 95%, or 99% of the residues of one nucleic acid sequence are complementary to residues of the other nucleic acid sequence. Sequences are said to be “substantially complementary” when at least 60% (e.g., at least 70%, at least 80%, or at least 90%) of the residues of one nucleic acid sequence are complementary to residues of the other nucleic acid sequence. In some embodiments, the biological sample is mounted on a first substrate and the substrate comprising the substrate of capture probes is a second substrate. In this configuration, one or more analytes or analyte derivatives (e.g., intermediate agents; e.g., ligation products) are then released from the biological sample and migrate to the second substrate comprising a substrate of capture probes. In some embodiments, the release and migration of the analytes or analyte derivatives to the second substrate comprising the substrate of capture probes occurs in a manner that preserves the original spatial context of the analytes in the biological sample. This method can be referred to as a sandwiching process, which is described, e.g., in U.S. Patent Application Pub. No. 2021/0189475 and PCT Pub. Nos. WO 2021/252747 A1, WO 2022/061152 A2, and WO 2022/140028 A1, each of which is herein incorporated by reference.

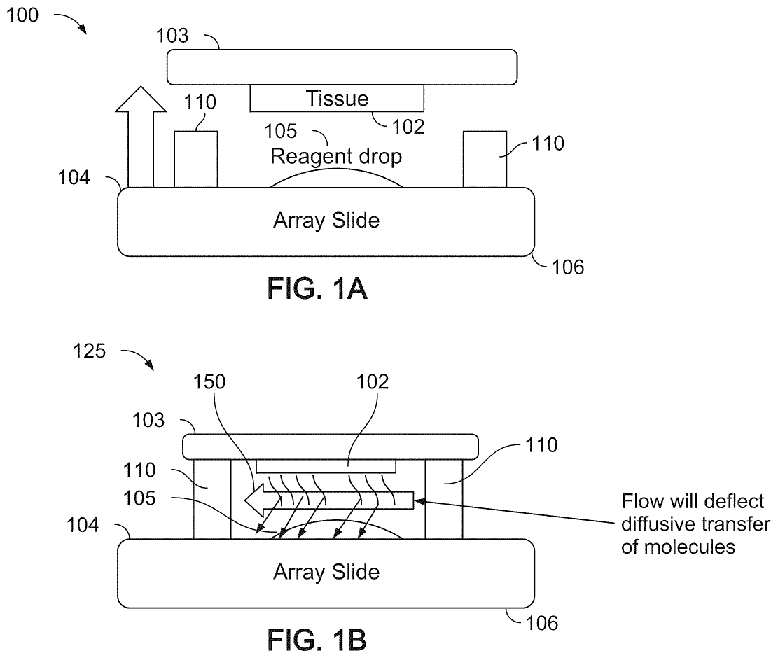

FIG. 1A shows an exemplary sandwiching process 100 where a first substrate (e.g., slide 103), including a biological sample 102, and a second substrate (e.g., slide 104 including a substrate having spatially barcoded capture probes 106) are brought into proximity with one another. As shown in FIG. 1A, a liquid reagent drop (e.g., permeabilization solution 105) is introduced on the second substrate in proximity to the capture probes 106 and in between the biological sample 102 and the second substrate (e.g., slide 104 including a substrate having spatially barcoded capture probes 106). The permeabilization solution 105 may release analytes or analyte derivatives (e.g., intermediate agents; e.g., ligation products) that can be captured by the capture probes of the substrate 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. Herein, the reagent medium may also comprise one or more of a monovalent salt, a divalent salt, ethylene carbonate, and/or glycerol. In some embodiments, analytes (e.g., mRNA transcripts) and/or analyte derivatives (e.g., intermediate agents; e.g., ligation products) of the biological sample 102 may release from the biological sample, and actively or passively migrate (e.g., diffuse) across the gap toward the capture probes on the substrate 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788 and U.S. Patent Application Pub. No. 2021/0189475, each of which is hereby incorporated by reference in its entirety.

As further shown, one or more spacers 110 may be positioned between the first substrate (e.g., slide 103) and the second substrate (e.g., slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns (μm) and about 1 mm (e.g., between about 2 μm and about 800 μm, between about 2 μm and about 700 μm, between about 2 μm and about 600 μm, between about 2 μm and about 500 μm, between about 2 μm and about 400 μm, between about 2 μm and about 300 μm, between about 2 μm and about 200 μm, between about 2 μm and about 100 μm, between about 2 μm and about 25 μm, or between about 2 μm and about 10 μm), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 μm. In some embodiments, the separation distance is less than 50 μm. In some embodiments, the separation distance is less than 25 μm. In some embodiments, the separation distance is less than 20 μm. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including a substrate 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate (e.g., slide 103), and the second substrate (e.g., slide 104) may reduce or prevent undesirable movement (e.g., convective movement) of transcripts and/or molecules during the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and a substrate alignment device). Further details on support devices, sample holders, sample handling apparatuses, or systems for implementing a sandwiching process are described in, e.g., U.S. Patent Application Pub. No. 2021/0189475 and PCT Publ. No. WO 2022/061152 A2, each of which is incorporated by reference in its entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

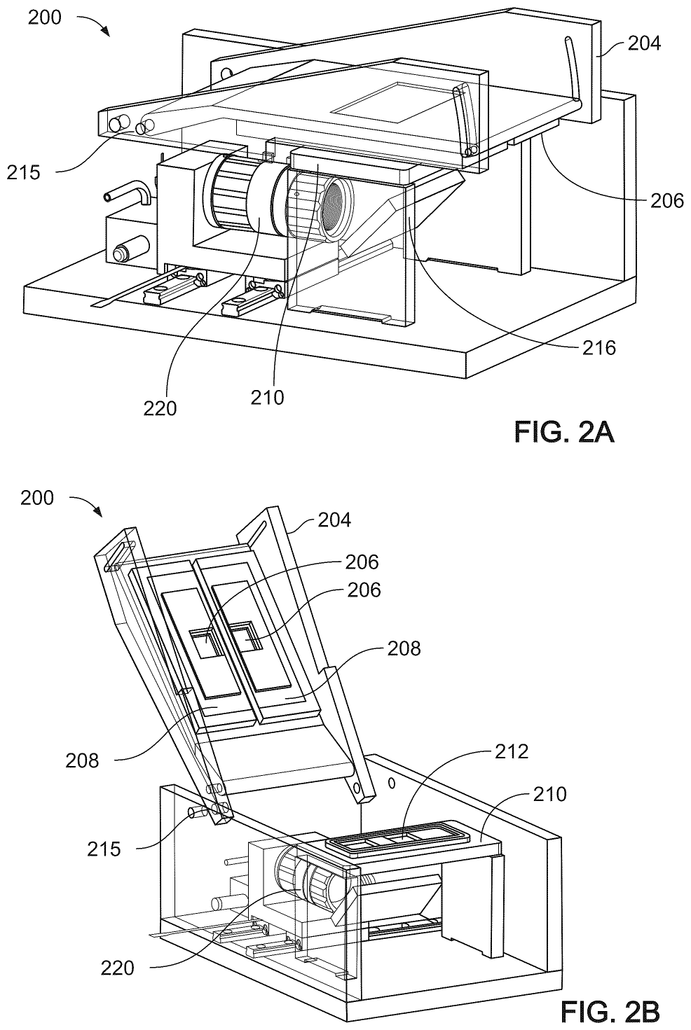

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded surface of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the substrate and may be processed for spatial analysis.

In some embodiments, during the permeabilization step, the image capture device 220 may capture images of the overlap area between the biological sample and the capture probes on the substrate 106. If more than one first substrates 206 and/or second substrates 212 are present within the sample handling apparatus 200, the image capture device 220 may be configured to capture one or more images of one or more overlap areas.

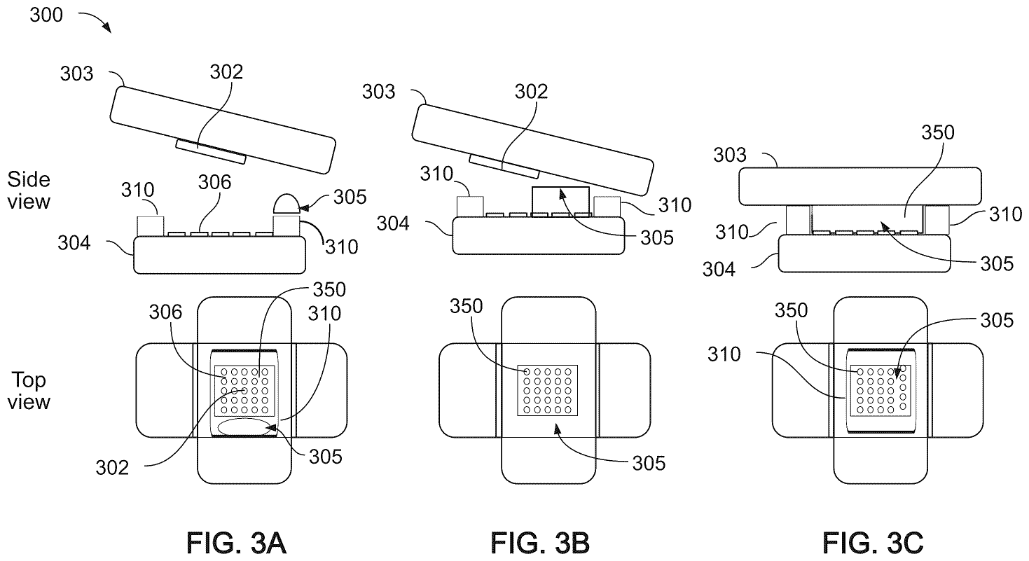

Provided herein are methods for delivering a fluid to a biological sample disposed on an area of a first substrate and a substrate disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right-hand side of side view, it should be understood that such depiction is not meant to be limiting as to the location of the reagent medium on the spacer.

FIG. 3B shows that as the first substrate lowers and/or as the second substrate rises, the dropped side of the first substrate (e.g., a side of the slide 303 angled toward the slide 304) may contact the reagent medium 305. The dropped side of the slide 303 may urge the reagent medium 305 toward the opposite direction (e.g., towards an opposite side of the spacer 310, towards an opposite side of the slide 303 relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

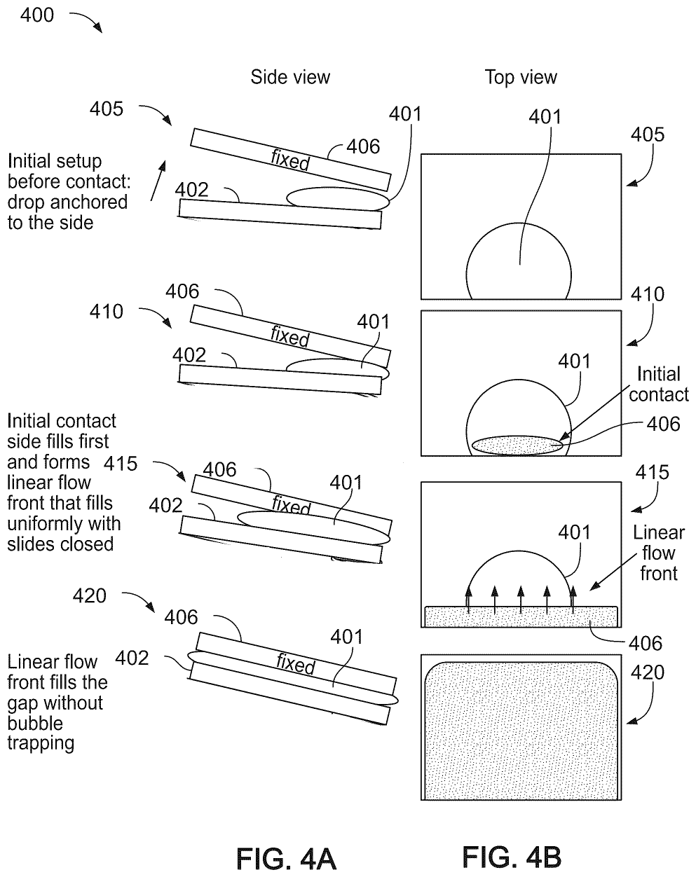

FIG. 4A is a side view of the angled closure workflow 400 in accordance with some exemplary implementations. FIG. 4B is a top view of the angled closure workflow 400 in accordance with some exemplary implementations. As shown at step 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills the gap between the two substrates 406 and 402 uniformly with the slides closed.

At step 415, the substrate 406 is further lowered toward the substrate 402 (or the substrate 402 is raised up toward the substrate 406) and the dropped side of the substrate 406 may contact and urge the reagent medium toward the side opposite the dropped side, thereby creating a linear or low curvature flow front that may prevent or reduce bubble trapping between the substrates.

At step 420, the reagent medium 401 fills the gap between the substrate 406 and the substrate 402. The linear flow front of the liquid reagent may be formed by squeezing the reagent medium 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, or methanol), cross-linking agents (e.g., paraformaldehyde), detergents (e.g., saponin, Triton X-100™, Tween-20™, SDS), and enzymes (e.g., trypsin or other proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and SDS. More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, elastase, and proteinase K. In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of SDS or a sodium salt thereof, proteinase K, pepsin, N-lauroylsarcosine, and RNase.

In some embodiments, the reagent medium comprises polyethylene glycol (PEG). In some embodiments, the PEG molecular weight is from about 2K to about 16K. In some embodiments, the PEG is about 2K, about 3K, about 4K, about 5K, about 6K, about 7K, about 8K, about 9K, about 10K, about 11K, about 12K, about 13K, about 14K, about 15K, or about 16K. In some embodiments, the PEG is present at a concentration from about 2% to about 25%, from about 4% to about 23%, from about 6% to about 21%, or from about 8% to about 20% (v/v).

In certain embodiments, a dried permeabilization reagent is applied or formed as a layer on the first substrate, the second substrate, or both prior to contacting the biological sample with the substrate. For example, a permeabilization reagent can be deposited in solution on the first substrate or the second substrate or both and then dried.

In some instances, the aligned portions of the biological sample and the substrate are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the substrate are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) to move out of a cell and towards a spatially-barcoded substrate (e.g., including spatially-barcoded capture probes). Another method is to cleave spatially-barcoded capture probes from a substrate and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Publication No. WO2020/176788 and/or U.S. Patent Application Publication No. 2020/0277663 regarding extended capture probes, which is herein incorporated by reference). In some cases, capture probes may be configured to form ligation products with a template (e.g., a DNA or RNA template, such as an analyte or an intermediate agent, or portion thereof), thereby creating ligation products that serve as proxies for the template.

As used herein, an “extended capture probe” refers to a capture probe having additional nucleotides added to a terminus (e.g., a 3′ or 5′ end) of the capture probe, thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using a reverse transcriptase. In some embodiments, the capture probe is extended using one or more DNA polymerases. In some embodiments, the extended capture probes include the sequence of the capture domain, the sequence of the spatial barcode of the capture probe, and the complementary sequence of the template used for extension of the capture probe.

In some embodiments, extended capture probes are amplified (e.g., in bulk solution or on the substrate) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II)(a) of PCT Publication No. WO2020/176788 and/or U.S. Patent Application Publication No. 2020/0277663, which is herein incorporated by reference. Analysis of captured analytes (and/or intermediate agents or portions thereof), for example, including sample removal, extension of capture probes using the captured analyte as a template, sequencing (e.g., of a cleaved extended capture probe and/or a cDNA molecule complementary to an extended capture probe), sequencing on the substrate (e.g., using, for example, in situ hybridization or in situ ligation approaches), temporal analysis, and/or proximity capture, is described in Section (II)(g) of PCT Publication No. WO2020/176788 and/or U.S. Patent Application Publication No. 2020/0277663, which is herein incorporated by reference. Some quality control measures are described in Section (II)(h) of PCT Publication No. WO2020/176788 and/or U.S. Patent Application Publication No. 2020/0277663, which is herein incorporated by reference.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660, each of which is herein incorporated by reference in its entirety.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up-regulated and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

For spatial analysis based methods, a substrate may function as a support for direct or indirect attachment of capture probes to features of the substrate. A “feature” is an entity that acts as a support or repository for various molecular entities used in spatial analysis. In some embodiments, some or all of the features in a substrate are functionalized for analyte capture. Exemplary substrates are described in Section (II)(c) of PCT Publication No. WO2020/176788 and/or U.S. Patent Application Publication No. 2020/0277663, which is herein incorporated by reference. Exemplary features and geometric attributes of a substrate can be found in Sections (II)(d)(i), (II)(d)(iii), and (II)(d)(iv) of PCT Publication No. WO2020/176788 and/or U.S. Patent Application Publication No. 2020/0277663, which is herein incorporated by reference.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads or wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of PCT Publication No. WO2020/176788 and/or U.S. Patent Application Publication No. 2020/0277663, which is herein incorporated by reference.

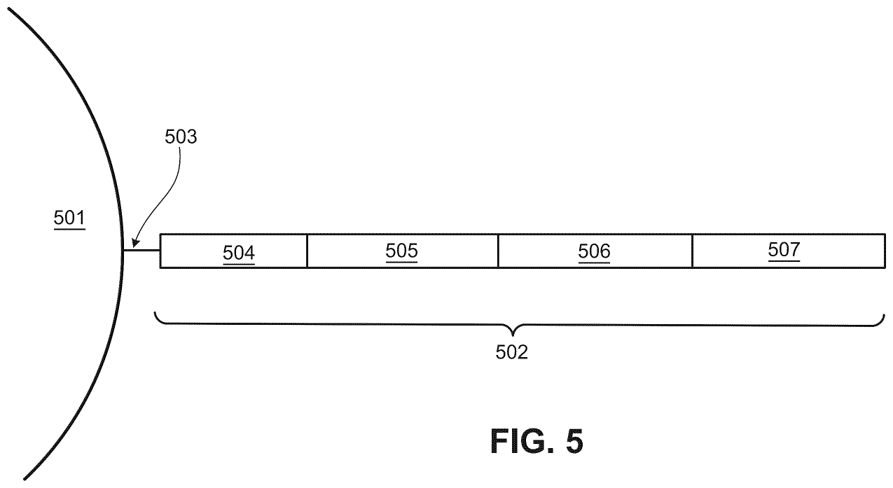

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that is useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to an analyte capture sequence present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

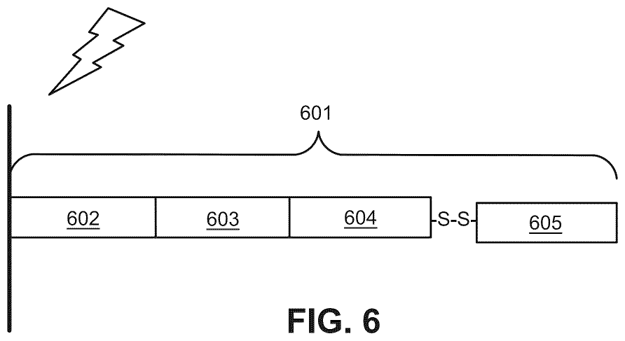

FIG. 6 is a schematic illustrating a cleavable capture probe, wherein the cleaved capture probe can enter into a non-permeabilized cell and bind to analytes within the cell. The capture probe 601 can contain a cleavage domain 602, a cell penetrating peptide 603, a reporter molecule 604, and a disulfide bond (—S—S—). 605 represents all other parts of a capture probe, for example, a spatial barcode and a capture domain.

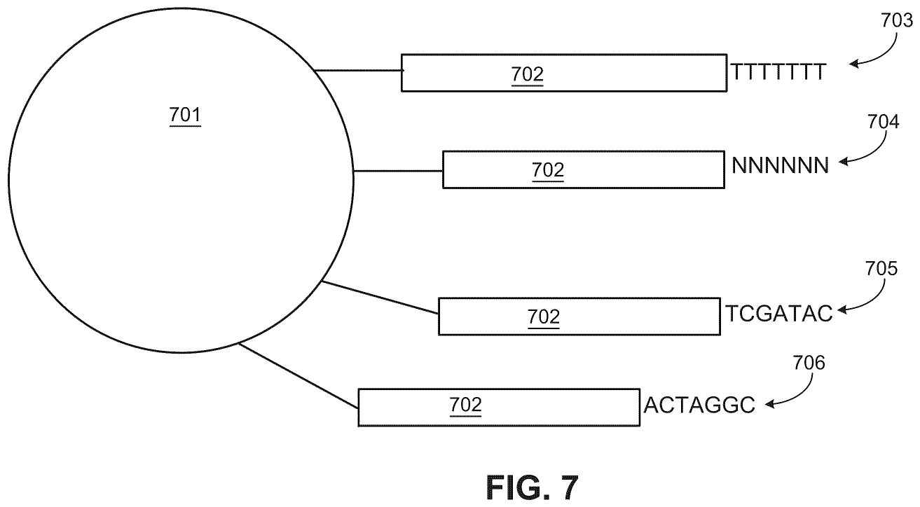

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may include four different types of spatially-barcoded capture probes, each type of spatially-barcoded capture probe possessing the spatial barcode 702. One type of capture probe associated with the feature can include the spatial barcode 702 in combination with a poly(T) capture domain 703, designed to capture mRNA target analytes. A second type of capture probe associated with the feature can include the spatial barcode 702 in combination with a random N-mer capture domain 704 for gDNA analysis. A third type of capture probe associated with the feature can include the spatial barcode 702 in combination with a capture domain complementary to the analyte capture agent of interest 705. A fourth type of capture probe associated with the feature can include the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and/or metabolites, and gDNA; (b) mRNA, accessible chromatin (e.g., ATAC-seq, DNase-seq, and/or MNase-seq), cell surface or intracellular proteins and/or metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature) change, or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequence 504 are common to all of the probes attached to a given feature. In some embodiments, the UMI sequence 506 of a capture probe attached to a given feature is different from the UMI sequence of a different capture probe attached to the given feature.

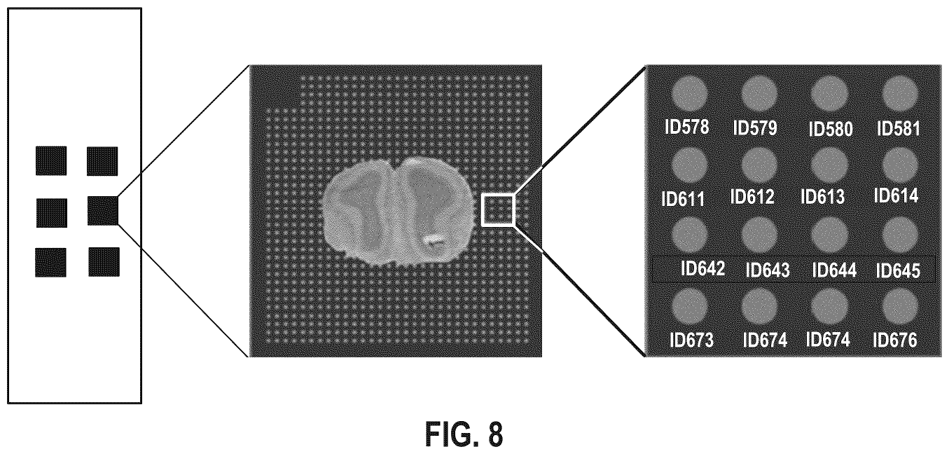

FIG. 8 depicts an exemplary arrangement of barcoded features within a substrate. From left to right, FIG. 8 shows (left) a slide including six spatially-barcoded regions, (center) an enlarged schematic of one of the six spatially-barcoded regions, showing a grid of barcoded features in relation to a biological sample, and (right) an enlarged schematic of one section of a substrate, showing the specific identification of multiple features within the substrate (e.g., labelled as ID578, ID579, ID580, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663, which is herein incorporated by reference.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663, which is herein incorporated by reference.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128, which is herein incorporated by reference in its entirety. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture probe binding domain (e.g., a poly(A) sequence or a non-homopolymeric sequence). After hybridization to the analyte, a ligase (e.g., a T4 RNA ligase (Rnl2), a PBCV-1 DNA Ligase or Chlorella virus DNA Ligase, a single-stranded DNA ligase, or a T4 DNA ligase) ligates the two oligonucleotides together, creating a ligation product.

After ligation, the ligation product is released from the analyte. In some instances, the ligation product is released using an endonuclease (e.g., RNase H). In some instances, the ligation product is removed using heat. In some instances, the ligation product is removed using KOH. The released ligation product can then be captured by capture probes (e.g., instead of direct capture of an analyte) on a substrate, optionally amplified, and sequenced, thus determining the location, and optionally, the abundance of the analyte in the biological sample.

In some instances, one or both of the oligonucleotides may hybridize to genomic DNA (gDNA), which can lead to false positive sequencing data from ligation events on gDNA (off target) in addition to the desired (on target) ligation events on target nucleic acids (e.g., mRNA). Thus, in some embodiments, the disclosed methods can include contacting the biological sample with a deoxyribonuclease (DNase). The DNase can be an endonuclease or exonuclease. In some embodiments, the DNase digests single-stranded and/or double-stranded DNA. Suitable DNases include, without limitation, a DNase I and a DNase II. Use of a DNase as described can mitigate false positive sequencing data from off target gDNA ligation events.

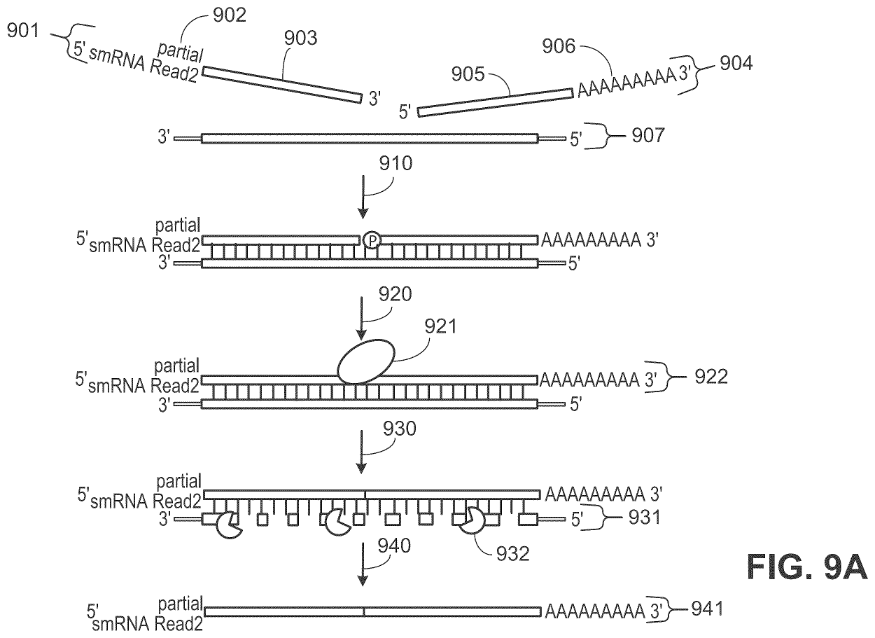

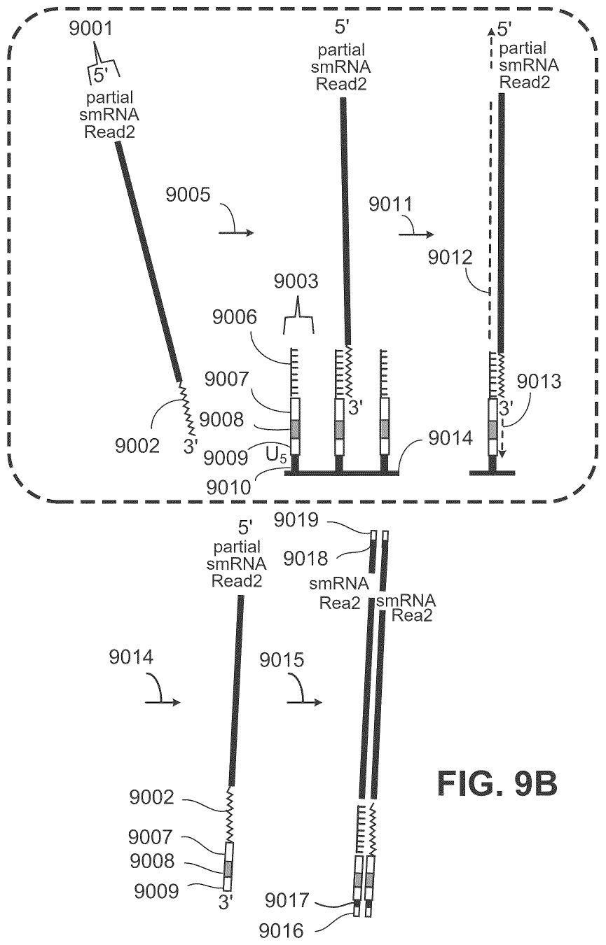

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly(A) sequence) 906, the first probe 901 and the second probe 904 hybridize 910 to an analyte 907. A ligase 921 ligates 920 the first probe 901 to the second probe 904, thereby generating a ligation product 922. The ligation product 922 is then released 930 from the analyte 931 by digesting the analyte 907 using an endoribonuclease 932. The sample is permeabilized 940 and the ligation product 941 is able to hybridize to a capture probe on the substrate. Methods and compositions for spatial detection using templated ligation have been described in PCT Publication. No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product 9001 specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily capture the ligation products (i.e., compared to no permeabilization). In some embodiments, polymerization (e.g., reverse transcription (RT)) reagents can be added to permeabilized biological samples. Incubation with the polymerization reagents can be used to extend the capture probes 9011 to produce spatially-barcoded full-length cDNA 9012 and 9013 from the captured ligation products (e.g., ligation products). The ligation products can be extended using the capture probe as a template to include a complement of the capture probe, thereby generating extended ligation products.

In some embodiments, the extended ligation products can be denatured 9014, released from the capture probe, and transferred (e.g., to a clean tube) for amplification and/or library construction. The spatially-barcoded ligation products can be amplified 9015 via PCR prior to library construction. P5 9016, i5 9017, i7 9018, and P7 9019 sequences can be used as sample indexes. The amplicons can then be sequenced using paired-end sequencing using TruSeq Read 1 and TruSeq Read 2 as sequencing primer sites.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Publication No. WO2020/176788 and/or Section (II)(b)(viii) U.S. Patent Application Publication No. 2020/0277663, which is herein incorporated by reference.

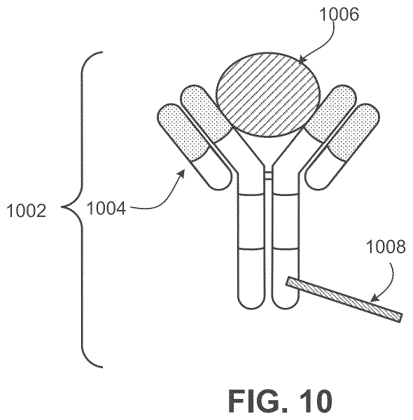

FIG. 10 is a schematic diagram of an exemplary analyte capture agent 1002 comprised of an analyte binding moiety 1004 and an analyte-binding moiety barcode domain 1008. The exemplary analyte binding moiety 1004 is capable of binding to an analyte 1006 and the analyte capture agent 1002 is capable of interacting with a spatially-barcoded capture probe. The analyte binding moiety 1004 can bind to the analyte 1006 with high affinity and/or with high specificity. The analyte capture agent 1002 can include: (i) an analyte binding moiety barcode domain 1008, which serves to identify the analyte binding moiety, and (ii) an analyte capture sequence, which can hybridize to at least a portion or an entirety of a capture domain of a capture probe. The analyte binding moiety 1004 can include a polypeptide and/or an aptamer. The analyte binding moiety 1004 can include an antibody or antibody fragment (e.g., an antigen-binding fragment).

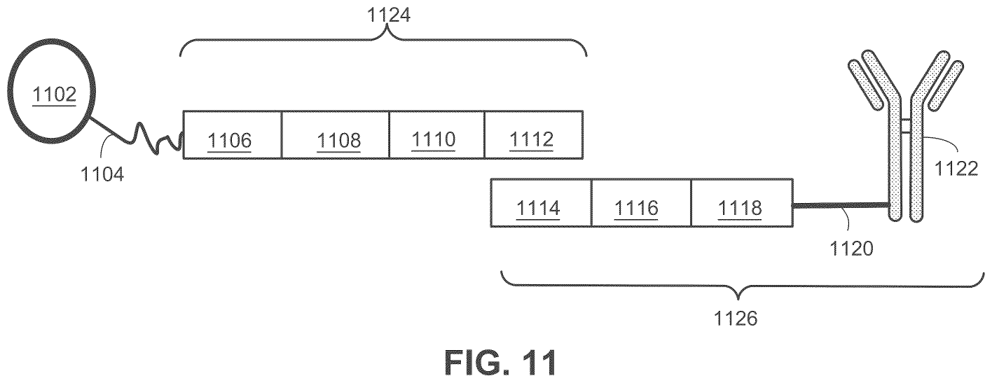

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequence 1106 and a UMI 1110, as described elsewhere herein. The capture probe can be affixed 1104 to a feature such as a bead 1102. The capture probe 1124 can also include a capture domain 1112 that is capable of binding to an analyte capture agent 1126. The analyte binding moiety barcode domain of the analyte capture agent 1126 can include functional sequence 1118, analyte binding moiety barcode 1116, and an analyte capture sequence 1114 that is capable of binding (e.g., hybridizing) to the capture domain 1112 of the capture probe 1124. The analyte capture agent 1126 can also include a linker 1120 that allows the analyte binding moiety barcode domain (e.g., including the functional sequence 1118, analyte binding moiety barcode 1116, and analyte capture sequence 1114) to couple to the analyte binding moiety 1122. In some embodiments, the linker 1120 is a cleavable linker. In some embodiments, the cleavable linker is a photo-cleavable linker, a UV-cleavable linker, chemical-cleavable, thermal-cleavable, or an enzyme cleavable linker. In some instances, the cleavable linker is a disulfide linker. A disulfide linker can be cleaved by use of a reducing agent, such as dithiothreitol (DTT), beta-mercaptoethanol (BME), or Tris (2-carboxyethyl) phosphine (TCEP).

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the captured analytes are associated with specific locations in a substrate of features on a substrate. For example, specific spatial barcodes can be associated with specific locations prior to substrate fabrication, and the sequences of the spatial barcodes can be stored (e.g., in a database) along with specific location information, so that each spatial barcode uniquely maps to a particular location.

Alternatively, specific spatial barcodes can be deposited at predetermined locations in a substrate of features during fabrication such that at each location, only one type of spatial barcode is present so that each spatial barcode is uniquely associated with a single feature of the substrate. Where necessary, the substrates can be decoded using any of the methods described herein so that spatial barcodes are uniquely associated with feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with a substrate feature location. In this manner, specific capture probes and captured analytes are associated with specific locations in the substrate of features. Each feature location represents a position relative to a coordinate reference point (e.g., a substrate location or a fiducial marker) of the substrate. Accordingly, each feature location has an “address” or location in the coordinate space of the substrate.

Some exemplary spatial analysis workflows are described in the Exemplary Embodiments section of PCT Publication No. WO2020/176788 and/or U.S. Patent Application Publication No. 2020/0277663, which is herein incorporated by reference. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663, which is herein incorporated by reference. See also, e.g., the Visium Spatial Gene Expression Reagent Kits User Guide (e.g., Rev F, dated January 2022) and/or the Visium Spatial Gene Expression Reagent Kits—Tissue Optimization User Guide (e.g., Rev E, dated February 2022), each of which is herein incorporated by reference in its entirety.

In some embodiments, spatial analysis can be performed using dedicated hardware and/or software, such as any of the systems described in Sections (II)(e)(ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320, which is herein incorporated by reference.

Suitable systems for performing spatial analysis can include components such as a chamber (e.g., a flow cell or a sealable, fluid-tight chamber) for containing a biological sample. The biological sample can be mounted, for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable, and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the substrate. Exemplary methods to detect the biological sample on a substrate are described in PCT Publication No. WO2021/102003 and/or U.S. Patent Application Publication No. 2021/0150707, each of which is incorporated herein by reference in its entirety.

Prior to transferring analytes from the biological sample to the substrate of features on the substrate, the biological sample can be aligned with the substrate. Alignment of a biological sample and a substrate of features including capture probes can facilitate spatial analysis, which can be used to detect differences in analyte presence and/or level within different positions in the biological sample, for example, to generate a three-dimensional map of the analyte presence and/or level. Exemplary methods to generate a two-dimensional and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in its entirety.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in its entirety. Fiducial markers can be used as a point of reference or measurement scale for alignment (e.g., to align a sample and a substrate, to align two substrates, to determine a location of a sample on a substrate relative to a fiducial marker) and/or for quantitative measurements of sizes and/or distances.

B. Spatial Detection of Exogenous Nucleic Acids and/or Microbes in a Biological Sample

The present disclosure features methods, compositions, and kits for determining the presence and/or location of target nucleic acids in a biological sample (e.g., any of the biological samples described herein). In some embodiments, the target nucleic acids are exogenous to the biological sample. For example, exogenous nucleic acids can be detected from bacterial, archaeal, viral, and/or fungal sources present in a biological sample, such as an animal sample, including human, mouse, rat, dog, cat, horse, goat, sheep, and rabbit. Thus, the disclosed methods, compositions, and kits can be used to determine the presence and/or location of one or more microbes (e.g., an archaeon, a bacterium, a fungus, a virus) in a biological sample.

Identifying exogenous organisms in a biological sample can improve understanding of the most effective methods to treat the exogenous organism. Further, understanding pathology of exogenous organisms, including for example, location of bacterial, viral, and/or fungal nucleic acids within a biological sample helps elucidate molecular mechanisms of infection and potential treatments and/or therapies.

In some embodiments, the methods, compositions, and kits described herein can also be used to determine the presence and/or location of endogenous target nucleic acids and/or other analytes (e.g., protein) from the biological sample, as more fully described herein.

Thus, disclosed herein are methods for determining a location of an endogenous and/or exogenous target nucleic acid in a biological sample. Also disclosed are methods for determining the presence and/or location of a microbe (e.g., an archaeon, a bacterium, a fungus, a virus) in a biological sample. Further disclosed are methods for determining the presence and/or location of a pathogenic microbe (e.g., an archaeon, a bacterium, a fungus, a virus) in a biological sample. Typically, the methods include providing a first substrate comprising a plurality of capture probes comprising capture domains and spatial barcodes, contacting the biological sample with a plurality of padlock probes, hybridizing the padlock probe to a target nucleic acid (e.g., an exogenous target nucleic acid, a nucleic acid from a microbe), ligating the ends of the extended padlock probe to each other, cleaving the ligated padlock probe, hybridizing the cleaved (linear) padlock probe to the capture domain of a capture probe on the first substrate, determining the sequence of (i) the spatial barcode, or a complement thereof, and (ii) all or a part of the sequence of the linear padlock probe, or a complement thereof; and using the sequences of (i) and (ii) to determine the location of the target nucleic acid in the biological sample or to determine the presence and/or location of a microbe in the biological sample.

In some embodiments, the methods described herein utilize padlock probes for detection of specific target nucleic acids and/or closely related target nucleic acids (e.g., target nucleic acids with conserved regions) and a substrate including a plurality of capture probes, where a capture probe within the plurality includes a spatial barcode and a capture domain. Padlock probes are advantageous since a single probe can be used to detect target nucleic acids in comparison with traditional templated ligation which requires two or more probes. Single-probe based assays can save costs when generating panels of probes and potentially improve specificity over multiple probe based assays. In some embodiments, the methods include using one padlock probe per target nucleic acid to be detected. In some embodiments, the methods include using two or more padlock probes per target nucleic acid to be detected. For example, in some embodiments, the methods include using 2, 3, 4, 5, 6, 7, 8, 9, 10, 11, 12, 13, 14, 15, 16, 17, 18, 19, 20 or more padlock probes per target nucleic acid to be detected.

Thus provided herein are methods for determining a location of a target nucleic acid in a biological sample including: a) contacting the biological sample with a plurality of padlock probes, where a padlock probe of the plurality of padlock probes includes a first sequence substantially complementary to the target nucleic acid, a capture probe binding domain, a cleavage site, and a second sequence substantially complementary to the target nucleic acid; b) hybridizing the padlock probe to the target nucleic acid; c) extending the second sequence substantially complementary to the target nucleic acid, thereby generating an extended padlock probe; d) ligating a first end of the extended padlock probe to a second end of the extended padlock probe, thereby generating a ligated padlock probe; e) cleaving the cleavage site of the ligated padlock probe, thereby generating a linear padlock probe; f) hybridizing the capture probe binding domain of the linear padlock probe to the capture domain of a capture probe on a first substrate, where the capture probe is included in a plurality of capture probes on the first substrate, the capture probe including: i) a spatial barcode and ii) the capture domain; and g) determining the sequence of (i) the spatial barcode, or a complement thereof, and (ii) all or a part of the sequence of the linear padlock probe, or a complement thereof; and using the sequences of (i) and (ii) to determine the location of the target nucleic acid in the biological sample.

Also provided herein are methods for determining a location of a target nucleic acid in a biological sample including: a) providing a first substrate including a plurality of capture probes, where a capture probe of the plurality of capture probes comprises: i) a spatial barcode and ii) a capture domain; b) contacting the biological sample with a plurality of padlock probes, where a padlock probe of the plurality of padlock probes includes a first sequence substantially complementary to the target nucleic acid, a capture probe binding domain, a cleavage site, and a second sequence substantially complementary to the target nucleic acid; c) hybridizing the padlock probe to the target nucleic acid; d) ligating the ends of the padlock probe to each other, thereby generating a ligated padlock probe; e) cleaving the cleavage site of the ligated padlock probe, thereby generating a linear padlock probe; f) hybridizing the capture probe binding domain of the linear padlock probe to the capture domain of the capture probe on the first substrate; and g) determining the sequence of (i) the spatial barcode, or a complement thereof, and (ii) all or a part of the sequence of the linear padlock probe, or a complement thereof; and using the sequences of (i) and (ii) to determine the location of the target nucleic acid in the biological sample.

Also provided herein are methods for determining a presence and/or location of a microbial target nucleic acid in a biological sample including: a) providing a first substrate including a plurality of capture probes, where a capture probe of the plurality of capture probes includes: i) a spatial barcode and ii) a capture domain; b) contacting the biological sample with a plurality of padlock probes, where a padlock probe of the plurality of padlock probes includes a first sequence substantially complementary to the microbial target nucleic acid, a capture probe binding domain, a cleavage site, and a second sequence substantially complementary to the microbial target nucleic acid; c) hybridizing the padlock probe to the microbial target nucleic acid; d) ligating ends of the extended padlock probe to each other, thereby generating a ligated padlock probe; e) cleaving the cleavage site of the ligated padlock probe, thereby generating a linear padlock probe; f) hybridizing the capture probe binding domain of the linear padlock probe to the capture domain of the capture probe on the substrate; and g) determining the sequence of (i) the spatial barcode and (ii) all or a part of the sequence of the linear padlock probe; and using the sequences of (i) and (ii) to determine the presence and/or location of the microbial target nucleic acid in the biological sample; optionally where the microbial target nucleic acid is derived from a microbe selected from the group including fungi, bacteria, archaea, or a combination thereof, optionally where the microbe is a pathogenic microbe.

As used herein a “padlock probe” is a single-stranded nucleic acid probe that includes two sequences that are substantially complementary to a target nucleic acid. The two sequences (i.e., the first sequence and the second sequence) are located at or near opposite ends of the probe. The padlock probe can be a linear molecule when not hybridized to a target nucleic acid. For example, the padlock probe can include a free first end (e.g., a 5′ end) and a free second end (e.g., a 3′ end). In some embodiments, ligating the ends of the padlock probe to each other in accordance of the disclosed methods includes ligating a free first end of the padlock probe to a free second end of the padlock probe. In some embodiments, ligating the ends of the padlock probe to each other in accordance of the disclosed methods includes ligating a 5′end of the padlock probe to a 3′end of the padlock probe. The padlock probe can include one or more additional domains (e.g., capture probe binding domains, cleavages sites, and/or functional domains) as further described herein. In some embodiments, the padlock probe includes DNA.

As used herein an “extended padlock probe” is a single-stranded padlock probe hybridized to a target nucleic acid, where either the first sequence substantially complementary to the target nucleic acid or the second sequence substantially complementary to the target nucleic acid is extended, e.g., via a polymerase (e.g., with any of the polymerases described herein). The extension process can also be referred to as “gap-filling” or “a gap-filling reaction.” Thus, the “extended padlock probe” includes a further sequence not part of the padlock probe prior to its extension.

As used herein a “ligated padlock probe” refers to both a padlock probe that has hybridized to adjacent sequences in a target nucleic acid and is ligated (e.g., no extension of the padlock probe occurs before ligation) and a padlock probe that has been extended and subsequently ligated. The ligated padlock probe can also be referred to as a “closed padlock probe” or a “circularized padlock probe.” Ligation can be performed enzymatically or chemically. For example, the padlock probe or extended padlock probe may be subjected to an enzymatic ligation reaction using a ligase (e.g., T4 RNA ligase (Rnl2), a SplintR ligase, PBCV-1 DNA ligase or Chlorella virus DNA ligase, or a T4 DNA ligase).

As used herein, a “linear padlock probe” or a “linearized padlock probe” refers to a ligated padlock probe that has been cleaved. For example, a ligated padlock probe (e.g., a circularized padlock probe) can include a cleavage site. When the cleavage site is cleaved, the ligated padlock probe becomes a molecule that is now linear (e.g., having free 5′ and 3′ ends) and no longer circularized.

As used herein an “extended linear padlock probe” refers to a linear padlock probe that has been further extended to include a sequence complement of a template. For example, an “extended linear padlock probe” encompasses a linear padlock probe that is hybridized to a capture probe on a substrate (e.g., a first substrate) and extended to include a complement of all or a portion of the capture probe. The extended linear padlock probe can include a capture probe binding domain (e.g., a sequence substantially complementary to the capture domain of a capture probe) that hybridizes to the capture domain of the capture probe. The captured linear padlock probe can be extended (i.e., via a polymerase) to incorporate complementary sequences of the capture probe. The resulting nucleic acid product is referred to as an “extended linear padlock probe.”

In some embodiments, the substrate includes one or more features. In some embodiments, features are directly or indirectly attached or fixed to a substrate. In some embodiments, the features are not directly or indirectly attached or fixed to a substrate, but instead, for example, are disposed within an enclosed or partially enclosed three-dimensional space (e.g., wells or divots). For example, the plurality of capture probes can be located on features on a substrate (e.g., a glass slide). In some embodiments, features include, but are not limited to, a spot, an inkjet spot, a masked spot, a pit, a post, a well, a ridge, a divot, a hydrogel pad, and a bead (e.g., a hydrogel bead). In some embodiments, the one or more features includes a well, a post, a ridge, a divot, or a bead. In some embodiments, the substrate includes a plurality of beads as disclosed elsewhere herein.

In some embodiments, the biological sample is disposed on the first substrate including the plurality of capture probes. In some embodiments, the biological sample is disposed on a second substrate. In some embodiments, the method includes aligning the second substrate including the biological sample with the first substrate, such that at least a portion of the biological sample is aligned with at least a portion of the first substrate (e.g., sandwiched as described herein).

In some embodiments, the capture probe includes one or more functional domains, a unique molecular identifier (UMI) (as described herein), a cleavage domain, or combinations thereof. In some embodiments, the unique molecular identifier is located 5′ to the capture domain in the capture probe. In some embodiments, the functional domain includes a sequencing specific site or a primer binding site. In some embodiments, the functional domain includes an amplification (e.g., PCR) sequence. A functional domain typically includes a functional nucleotide sequence for a downstream analytical step in the overall analysis procedure.

In some embodiments, the capture domain includes a homopolymeric sequence. In some embodiments, the homopolymeric sequence includes a poly(T) sequence. In some embodiments, the poly(T) sequence includes about 10, 15, 20, 25, 30, or more nucleotides (e.g., Ts). In some embodiments, the capture domain includes a fixed sequence. As used herein, a “fixed sequence” is a non-random sequence. For example, the capture domain and the capture probe binding domain can be any sequence as long as the sequences are substantially complementary to one another to facilitate hybridization.

In some embodiments, the capture domain includes a random sequence, e.g., a random hexamer, a random decamer.

In some embodiments, the hybridizing in step (c) includes hybridizing the first sequence substantially complementary to the target nucleic acid and the second sequence substantially complementary to the target nucleic acid of the padlock probe to the target nucleic acid.

In some embodiments, the first sequence substantially complementary to the target nucleic acid and the second sequence substantially complementary to the target nucleic acid hybridize to conserved regions of the target nucleic acid. In some embodiments, the first sequence substantially complementary to the target nucleic acid and the second sequence substantially complementary to the target nucleic acid flank a variable region of the target nucleic acid.

In some instances, the complementary sequences in the target nucleic acid to which the first sequence and the second sequence of the padlock probe hybridize are adjacent to one another (e.g., directly abutting each other). In some embodiments, the complementary sequences in the target nucleic acid to which the first sequence and the second sequence of the padlock probe hybridize are spaced apart. In some embodiments, the complementary sequences in the target nucleic acid to which the first sequence and the second sequence of the padlock probe hybridize are 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, about 125, or about 150 nucleotides away from each other. Gaps between the first and second sequences substantially complementary to the target nucleic acid can be filled prior to coupling (e.g., ligation), e.g., using, for example, dNTPs in combination with a polymerase such as polymerase mu, DNA polymerase, RNA polymerase, reverse transcriptase, VENT polymerase, Taq polymerase, and/or any combinations, derivatives, and variants (e.g., engineered mutants) thereof. In some embodiments, when the first and second sequences substantially complementary to the target nucleic acid are separated from each other by one or more nucleotides upon hybridizing to the target nucleic acid, deoxyribonucleotides are used to extend and couple (e.g., ligate) the first and/or second sequences of the padlock probe.

In some embodiments, hybridizing the padlock probe to the target nucleic acid and extending the second sequence substantially complementary to the target nucleic acid occur sequentially. For example, in some embodiments hybridizing the padlock probe to the target nucleic acid can be performed in an overnight reaction and extending the second sequence substantially complementary to the target nucleic acid is performed subsequently. In some embodiments, hybridizing the padlock probe to the target nucleic acid and extending the second sequence substantially complementary to the target nucleic acid occur simultaneously.

In some embodiments, ligating the ends (e.g., a 5′ end and 3′ end) of the padlock probe to each other includes chemical or enzymatic ligation. In some embodiments, ligating the ends of the padlock probe includes ligating a first end of the padlock probe to a second end of the padlock probe. In some embodiments, ligating the ends of the padlock probe includes ligating a 5′ end of the padlock probe to a 3′ end of the padlock probe. In some embodiments, ligating the ends of the padlock probe includes circularizing the padlock probe (e.g., using a CircLigase).

In some embodiments, ligating the ends (e.g., a 5′ end and 3′ end) of the extended padlock probe to each other includes chemical or enzymatic ligation. In some embodiments, ligating the ends of the extended padlock probe includes ligating a first end of the extended padlock probe to a second end of the extended padlock probe. In some embodiments, ligating the ends of the extended padlock probe includes ligating a 5′ end of the extended padlock probe to a 3′ end of the extended padlock probe. In some embodiments, ligating the ends of the extended padlock probe includes circularizing the extended padlock probe (e.g., using a CircLigase).

In some embodiments, ligating the ends of the padlock probe or extended padlock probe is performed using a ligase, a ligase selected from the group consisting of: Tth DNA ligase, Taq DNA ligase, Thermococcus sp. DNA ligase, PBCV-1 DNA ligase, CircLigase™ II, and Chlorella virus DNA ligase.

In some embodiments, the padlock probe includes in a 5′ to 3′ direction: (i) a first sequence substantially complementary to the target nucleic acid; (ii) a capture probe binding domain; (iii) a cleavage site; and (iv) a second sequence substantially complementary to the target nucleic acid.

In some embodiments, the padlock probe includes a functional domain. In some embodiments, the functional domain is a sequencing specific site or a primer binding site. In some embodiments, the functional domain is disposed 3′ to the cleavage site. In some embodiments, the functional domain is disposed 5′ to the second sequence substantially complementary to the target nucleic acid. In some embodiments, the functional domain is disposed both 3′ to the cleavage site and 5′ to the second sequence substantially complementary to the target nucleic acid.

In some embodiments, the method includes releasing the ligated padlock probe from the target nucleic acid. In some embodiments, the releasing is performed prior to hybridizing the capture probe binding domain of the linear padlock probe to the capture domain of the capture probe (e.g., as recited in step (g) of some of the methods disclosed herein). In some embodiments, the releasing includes the use of an endoribonuclease (e.g., an RNase). In some embodiments, the releasing includes use of one or more RNases. In some embodiments, the one or more RNases includes RNase A, RNase C, RNase H, and/or RNase I.

In some embodiments, RNase H includes one or both of RNase H1 and RNase H2. RNase H is an endoribonuclease that specifically hydrolyzes the phosphodiester bonds of RNA, when hybridized to DNA. RNase H is part of a conserved family of ribonucleases which are present in many different organisms. There are two primary classes of RNase H: RNase H1 and RNase H2. Retroviral RNase H enzymes are similar to the prokaryotic RNase H1. All of these enzymes share the characteristic that they are able to cleave the RNA component of an RNA:DNA heteroduplex. RNase H includes but is not limited to RNase HII from Pyrococcus furiosus, RNase HII from Pyrococcus horikoshi, RNase HI from Thermococcus litoralis, RNase HI from Thermus thermophilus, RNase HI from E. coli, or RNase HII from E. coli.

In some embodiments, the releasing includes the use of KOH. In some embodiments, the releasing includes the use of heat.

In some embodiments, extending the second sequence substantially complementary to the target nucleic acid includes use of a polymerase. In some embodiments, the polymerase is a DNA polymerase. In some embodiments, the polymerase is a reverse transcriptase. In some embodiments, the first sequence substantially complementary to the target nucleic acid is extended (e.g., extended with a polymerase). For example, depending on the orientation of the padlock probe hybridized to the target nucleic acid, either the first sequence substantially complementary to the target nucleic acid or the second sequence substantially complementary to the target nucleic acid can be extended (e.g., a free 3′ end of either the first sequence or the second sequence can be extended).

In some embodiments, the padlock probe includes about 200 nucleotides to about 500 nucleotides (e.g., about 200, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495 or more nucleotides). In some embodiments, the padlock probe includes about 250 nucleotides to about 450 nucleotides. In some embodiments, the padlock probe includes about 300 nucleotides.

In some embodiments, ligating the ends of the extended padlock probe is performed using a ligase selected from the group consisting of: a single-stranded DNA ligase, Tth DNA ligase, Taq DNA ligase, Thermococcus sp. DNA ligase, PBCV-1 DNA Ligase, and Chlorella virus DNA Ligase.

In some embodiments, the cleavage site is a restriction enzyme site. In some embodiments, the restriction enzyme site is cleaved by one or more restriction enzymes. In some embodiments the cleavage site (e.g., restriction enzyme site) is designed such that it is present in the padlock probe but not in the plurality of capture probes on the substrate. In some embodiments, the cleavage site includes a sequence that is recognized by one or more enzymes capable of cleaving a nucleic acid molecule, e.g., capable of breaking the phosphodiester linkage between two or more nucleotides. A bond can be cleavable via other nucleic acid molecule targeting enzymes, such as restriction enzymes (e.g., restriction endonucleases). For example, the cleavage site can include a restriction endonuclease (restriction enzyme) recognition sequence or restriction enzyme site. In some embodiments, the restriction enzyme site is cleaved by one or more restriction enzymes.

Any suitable restriction endonuclease cleavage site (i.e., restriction site) can be used in the methods described herein. Cleavage methods and procedures for selecting restriction enzymes for cutting nucleic acid at specific sites are well known to the skilled artisan. For example, many suppliers of restriction enzymes provide information on conditions and types of DNA sequences cut by specific restriction enzymes, including New England BioLabs, Pro-Mega Biochems, Boehringer-Mannheim, and the like. Nucleic acid to be cleaved often is/are free of certain contaminants such as phenol, chloroform, alcohol, EDTA, detergents, or excessive salts, all of which can interfere with restriction enzyme activity, in certain embodiments.

Restriction enzymes (i.e., restriction endonucleases) are traditionally classified into three types on the basis of subunit composition, cleavage position, sequence-specificity and cofactor-requirements. However, amino acid sequencing has uncovered extraordinary variety among restriction enzymes and revealed that at the molecular level there are many more than three different kinds.

Type I enzymes are complex, multi-subunit, combination restriction-and-modification enzymes that cut DNA at random far from their recognition sequences. Originally thought to be rare, we now know from the analysis of sequenced genomes that they are common. Type I enzymes are of considerable biochemical interest, but they have little practical value since they do not produce discrete restriction fragments or distinct gel-banding patterns.

Type II enzymes cut DNA at defined positions close to or within their recognition sequences. They produce discrete restriction fragments and distinct gel banding patterns, and they are the only class used in the laboratory for DNA analysis and gene cloning. Type II enzymes frequently differ so utterly in amino acid sequence from one another, and indeed from every other known protein, that they likely arose independently in the course of evolution rather than diverging from common ancestors.

The most common type II enzymes are those like HhaI, HindIII and NotI that cleave DNA within their recognition sequences. Enzymes of this kind are available commercially. Most recognize DNA sequences that are symmetric because they bind to DNA as homodimers, but a few, (e.g., BbvCI:CCTCAGC) recognize asymmetric DNA sequences because they bind as heterodimers. Some enzymes recognize continuous sequences (e.g., EcoRI:GAATTC) in which the two half-sites of the recognition sequence are adjacent, while others recognize discontinuous sequences in which the half-sites are separated. Cleavage leaves a 3′-hydroxyl on one side of each cut and a 5′-phosphate on the other. They require only magnesium for activity and the corresponding modification enzymes require only S-adenosylmethionine. They tend to be small, with subunits in the 200-350 amino acid range.

The next most common type II enzymes, usually referred to as ‘type IIs’ are those like FokI and AlwI that cleave outside of their recognition sequence to one side. These enzymes are intermediate in size, 400-650 amino acids in length, and they recognize sequences that are continuous and asymmetric. They comprise two distinct domains, one for DNA binding and the other for DNA cleavage. They are thought to bind to DNA as monomers for the most part, but to cleave DNA cooperatively, through dimerization of the cleavage domains of adjacent enzyme molecules. For this reason, some type s enzymes are much more active on DNA molecules that contain multiple recognition sites. A wide variety of Type IAS restriction enzymes are known, and such enzymes have been isolated from bacteria, phage, archaebacterial and viruses of eukaryotic algae and are commercially available (Promega, Madison Wis.; New England Biolabs, Beverly, Mass.). Examples of Type IBS restriction enzymes that may be used with methods described herein include but are not limited to enzymes such as those listed in Table 1.

TABLE 1
Examples of Type IIS restriction enzymes

| | Recognition/Cleavage | |
|---|---|---|
| Enzyme-Source | site | Supplier |
| Alw I-Acinetobacter lwoffii | GGATC(4/5) | NE Biolabs |
| Alw26 I-Acinetobacter lwoffi | GTCTC(1/5) | Promega |
| Bbs I-Bacillus laterosporus | GAAGAC(2/6) | NE Biolabs |
| Bbv I-Bacillus brevis | GCAGC(8/12) | NE Biolabs |
| BceA I-Bacillus cereus 1315 | IACGGC(12/14) | NE Biolabs |
| Bmr I-Bacillus megaterium | CTGGG(5/4) | NE Biolabs |
| Bsa I-Bacillus | GGTCTC(1/5) | NE Biolabs |
| stearothermophilus 6-55 | | |
| Bst71 I-Bacillus | GCAGC(8/12) | Promega |
| stearothermophilus 71 | | |
| BsmA I-Bacillus | GTCTC(1/5) | NE Biolabs |
| stearothermophilus A664 | | |
| BsmB I-Bacillus | CGTCTC(1/5) | NE Biolabs |
| stearothermophilus B61 | | |
| BsmF I-Bacillus | GGGAC(10/14) | NE Biolabs |
| stearothermophilus F | | |
| BspM I-Bacillus species M | ACCTGC(4/8) | NE Biolabs |
| BspQI-Bacillus spaericus | GCTCTTC(1/4) | NE Biolabs |
| Ear I-Enterobacter aerogenes | CTCTTC(1/4) | NE Biolabs |
| Fau I-Flavobacterium aquatile | CCCGC(4/6) | NE Biolabs |
| Fok I-Flavobacterium | GGATG(9/13) | NE Biolabs |
| okeonokoites | | |
| Hga I-Haemophilus gallinarum | GACGC(5/10) | NE Biolabs |
| Ple I-Pseudomonas lemoignei | GAGTC(4/5) | NE Biolabs |
| Sap I-Saccharopolyspora | GCTCTTC(1/4) | NE Biolabs |
| species | | |
| SfaN I-Streptococcus | GCATC(5/9) | NE Biolabs |
| faecalis ND547 | | |
| Sth 132 I-Streptococcus | CCCG(4/8) | Gene |
| thermophilus ST132 | | 195: 201-206 (1997) |

A third major kind of type II enzyme more properly referred to as “type IV” are large, combination restriction-and-modification enzymes, 850-1250 amino acids in length, in which the two enzymatic activities reside in the same protein chain. These enzymes cleave outside of their recognition sequences; those that recognize continuous sequences (e.g., Eco57I: CTGAAG) cleave on just one side; those that recognize discontinuous sequences cleave on both sides releasing a small fragment containing the recognition sequence. The amino acid sequences of these enzymes are varied but their organization are consistent. They comprise an N-terminal DNA-cleavage domain joined to a DNA-modification domain and one or two DNA sequence-specificity domains forming the C-terminus, or present as a separate subunit. When these enzymes bind to their substrates, they switch into either restriction mode to cleave the DNA, or modification mode to methylate it.

As discussed above, the length of restriction recognition sites varies. For example, the enzymes EcoRI, SacI and SstI each recognize a 6 base-pair (bp) sequence of DNA, whereas NotI recognizes a sequence 8 bp in length, and the recognition site for Sau3AI is only 4 bp in length. Length of the recognition sequence dictates how frequently the enzyme will cut in a random sequence of DNA. Enzymes with a 6 bp recognition site will cut, on average, every 4⁶ or 4096 bp; a 4 bp recognition site will occur roughly every 256 bp.

Different restriction enzymes can have the same recognition site—such enzymes are called isoschizomers. For example, the recognition sites for SacI and SstI are identical. In some cases, isoschizomers cut identically within their recognition site, but sometimes they do not. Isoschizomers often have different optimum reaction conditions, stabilities, and costs, which may influence the decision of which to use.

Restriction recognition sites can be unambiguous or ambiguous. The enzyme BamHI recognizes the sequence GGATCC and no others; therefore, it is considered “unambiguous.” In contrast, HinfI recognizes a 5 bp sequence starting with GA, ending in TC, and having any base between. HinfI has an ambiguous recognition site. XhoII also has an ambiguous recognition site: Py stands for pyrimidine (T or C) and Pu for purine (A or G), so XhoII will recognize and cut sequences of AGATCT, AGATCC, GGATCT and GGATCC.

The recognition site for one enzyme may contain the restriction site for another. For example, note that a BamHI recognition site contains the recognition site for Sau3AI. Consequently, all BamHI sites will cut with Sau3AI. Similarly, one of the four possible XhoII sites will also be a recognition site for BamHI and all four will cut with Sau3AI.

Most recognition sequences are palindromes-they read the same forward (5′ to 3′ on the top strand) and backward (5′ to 3′ on the bottom strand). Most restriction enzymes bind to their recognition site as dimers (pairs).

In some embodiments, a complementary oligonucleotide can hybridize to a nucleic acid sequence present in the padlock probe, thereby generating a restriction site.

In some embodiments, the cleavage site includes a poly(U) sequence which can be cleaved by a mixture of Uracil DNA glycosylase (UDG) and the DNA glycosylase-lyase Endonuclease VIII, commercially known as the USER™ enzyme. In some embodiments, the cleavage site is an invader Lock (iLock) cleavage site.

In some embodiments, the cleavage site is a photo-cleavable site or an ultraviolet (UV)-cleavable site.

In some embodiments, the method includes extending the capture probe using the linear padlock probe as a template, thereby generating an extended capture probe. In some embodiments, the method includes extending the linear padlock probe using the capture probe as a template, thereby generating an extended linear padlock probe. In some embodiments, the method includes both extending the capture probe using the linear padlock probe as a template and extending the linear padlock probe using the capture probe as a template. Extension of either the capture probe and/or the linear padlock probe can be performed with a polymerase (e.g., a DNA polymerase).

In some embodiments, the determining step includes sequencing. In some embodiments, the sequencing includes high-throughput sequencing. In some embodiments, the determining step includes fluorescent hybridization.

In some embodiments, the biological sample is derived from a mammal, a plant, a nematode, or a fungus.

In some embodiments, the target nucleic acid is endogenous to the biological sample. In some embodiments, the target nucleic acid is RNA. Non-limiting examples of RNA include various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than or equal to 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA.

In some embodiments, the target nucleic acid is exogenous to the biological sample. In some embodiments, the target nucleic acid is a bacterial, viral, fungal, or archaeal nucleic acid (e.g., DNA or RNA). In some embodiments, the target nucleic acid is bacterial RNA (See e.g., Giuliano M. G. and Engl C., The Lifecyle of Ribosomal RNA in Bacteria, RNA Damage and Repair, Chapter 2 (2021), the content of which is incorporated herein by reference in its entirety). In some embodiments the target nucleic acid is fungal RNA (See e.g., Banos S., et al., A comprehensive fungi-specific 18S rRNA gene sequence primer toolkit suited for diverse research issues and sequencing platforms, BMC Microbiology, 18 (190) (2018), the content of which is incorporated herein by reference in its entirety). In some embodiments the target nucleic acid is archaeal RNA. In some embodiments the target nucleic acid is viral RNA.

In some embodiments, the bacterial RNA is bacterial ribosomal RNA. In some embodiments, the bacterial ribosomal RNA is 16S ribosomal RNA or 5S ribosomal RNA. In some embodiments, the fungal RNA is 18S ribosomal RNA or internal transcribed spacer (ITS) region ribosomal RNA.

In some embodiments, the padlock probe comprises a sequence at least 90%, 95%, 96%, 97%, 98%, or 99% identical to SEQ ID NO: 1. In some embodiments, the padlock probe is SEQ ID NO: 1:

TTACCGCGGCKGCTGRCACAAAAAAAAAAAAAAAAAAA
AAAAAAAAAAANNNNGAAGAGCCCTTGGCACCCGAGAA
TTCCAGGACTACNVGGGTWTCTAAT,

where N=any nucleotide; K=guanine or thymine; R=adenine or guanine; V=guanine, cytosine, or adenine; W=adenine or thymine.

The disclosed methods can be used for spatial analysis of microbial nucleic acids in wider context of endogenous gene expression in the biological sample. For example, the methods can be used to assay global gene expression or targeted gene expression in a biological sample in combination with exogenous (e.g., microbial) nucleic acid assessment. Thus, in some embodiments, the method includes hybridizing both an endogenous target nucleic acid and a linear padlock probe (a proxy of an exogenous target nucleic acid) to a first capture domain of a first capture probe and a second capture domain of second capture probe on the substrate. In some embodiments, the method includes hybridizing both a proxy of an endogenous target nucleic acid (e.g., a ligation product or connected probe) and a linear padlock probe (a proxy of the exogenous target nucleic acid) to a first capture domain of a first capture probe and a second capture domain of second capture probe on the substrate. In some embodiments, the method further includes extending the first or second capture probe using the endogenous target nucleic acid as a template. In some embodiments, the method further includes extending the first or second capture probe using the ligation product or connected probe as a template and/or extending the ligation product or connected probe using the first or second capture probe as a template.

In some embodiments, the method includes determining the location of the endogenous target nucleic acid and the exogenous target nucleic acid in the biological sample. In some embodiments, the method further includes determining the sequence of (iii) a second spatial barcode comprised in the second capture probe, or a complement thereof, and (iv) all or a part of the sequence of the endogenous target nucleic acid, or a complement thereof; and using the sequences of (iii) and (iv) to determine the location of the endogenous target nucleic acid in the biological sample. In some embodiments, the method further includes determining the sequence of (iii) a second spatial barcode comprised in the second capture probe, or a complement thereof, and (iv) all or a part of the ligation product (connected probe), or a complement thereof; and using the sequences of (iii) and (iv) to determine the location of the endogenous target nucleic acid in the biological sample. In some embodiments, the determining includes sequencing.

In some embodiments, the method includes imaging the biological sample. In some embodiments, the biological sample is visualized or imaged using bright field microscopy. In some embodiments, the biological sample is visualized or imaged using fluorescence microscopy. Additional methods of visualization and imaging are known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy.

In some embodiments, the method includes staining the biological sample. In some embodiments, the staining includes use of immunofluorescence, immunohistochemistry, and/or hematoxylin and/or eosin. In some embodiments, the staining includes a fluorescent antibody directed to a target analyte (e.g., cell surface or intracellular proteins) in the biological sample. In some embodiments, the staining includes an immunohistochemistry stain directed to a target analyte (e.g., cell surface or intracellular proteins) in the biological sample. In some embodiments, the staining includes a chemical stain, such as hematoxylin and eosin (H&E) or periodic acid-Schiff (PAS). In some embodiments, staining the biological sample includes the use of a biological stain including, but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof. In some embodiments, significant time (e.g., days, months, or years) can elapse between staining and/or imaging the biological sample.

In some embodiments, the staining includes the use of a detectable label selected from the group consisting of a radioisotope, a fluorophore, a chemiluminescent compound, a bioluminescent compound, or a combination thereof.

In some embodiments, the method includes migrating the linear padlock probe or ligation product from the biological sample to the substrate. In some embodiments, migrating the linear padlock probe or ligation product includes passive migration (e.g., diffusion). In some embodiments, migrating the linear padlock probe or ligation product includes active migration (e.g., electrophoresis).

In some embodiments, the method includes permeabilizing the biological sample. Permeabilization of a biological sample can occur on a substrate where the substrate is aligned with the substrate including the plurality of spatially barcoded capture probes such that at least a portion of the biological sample is aligned with at least a portion of the substrate including the plurality of spatially barcoded capture probes or directly on the substrate including the plurality of spatially barcoded capture probes. In some embodiments, permeabilizing includes use of a protease. In some embodiments, the protease includes one or more of pepsin, proteinase K, cellulase, and collagenase. In some embodiments, the protease includes proteinase K.

In some embodiments, cleaving the cleavage site of the ligated padlock probe, releasing the ligated padlock probe from the target nucleic acid, and permeabilizing the biological sample are performed simultaneously. In some embodiments, cleaving the cleavage site of the ligated padlock probe, releasing the ligated padlock probe from the target nucleic acid, and permeabilizing the biological sample are performed sequentially. In some embodiments, cleaving the cleavage site of the ligated padlock probe and releasing the ligated padlock probe from the target nucleic acid are performed at the same time (e.g., in one step), while permeabilizing the biological sample is performed separately (e.g., prior to cleaving and/or linearizing the ligated padlock probe).

In some embodiments, the biological sample is a tissue sample. In some embodiments, the tissue sample is a fixed tissue sample. In some embodiments, the fixed tissue sample is a methanol-fixed tissue sample, an acetone-fixed tissue sample, a paraformaldehyde-fixed tissue sample, or a formalin-fixed paraffin-embedded tissue sample. In some embodiments, the tissue sample is a fresh-frozen tissue sample.

In some embodiments, the biological sample is a tissue section. In some embodiments, the tissue section is a fixed tissue section. In some embodiments, the fixed tissue section is a methanol-fixed tissue section, an acetone-fixed tissue section, a paraformaldehyde-fixed tissue section, or a formalin-fixed paraffin-embedded tissue section. In some embodiments, the tissue section is a fresh-frozen tissue section.

FFPE samples generally are heavily cross-linked and fragmented, and therefore this type of sample allows for limited RNA recovery using conventional detection techniques.

In some embodiments, the FFPE sample or section is deparaffinized, permeabilized, equilibrated, and blocked before target probe oligonucleotides are added. In some embodiments, deparaffinization includes using xylenes. In some embodiments, deparaffinization includes multiple washes with xylenes. In some embodiments, deparaffinization includes multiple washes with xylenes followed by removal of xylenes using multiple rounds of graded alcohol followed by washing the sample with water. In some aspects, the water is deionized water.

In some embodiments, the biological sample is plant sample. In some embodiments, the plant sample is derived from a crop plant selected from the group consisting of wheat, rice, maize, soybean, sunflower, sorghum, rape, alfalfa, cotton, barley, millet, sugar cane, tomato, tobacco, cassava, and potato. In some embodiments, the plant sample is derived from Arabidopsis thaliana. In some embodiments, the plant sample is a seed, a leaf, a stem or other region of a plant.

In some embodiments, the biological sample is an animal tissue sample or tissue section (e.g., human, mouse, or rat) and the target nucleic acid is bacterial, fungal, viral or archaeal.

In some embodiments, the biological sample is a plant tissue sample or tissue section and the target nucleic acid is bacterial, fungal, viral or archaeal.

In some embodiments, the location of two or more (e.g., 10, 100, 500, 1000, 5000, or more) target nucleic acids are determined in the biological sample. In some embodiments, contacting the biological sample with the plurality of padlock probes includes contacting the biological sample with about 100 to about 5,000 padlock probes (e.g., about 200, 300, 400, 500, 1,000, 1,500, 2,000, 2,500, 3,000, 3,500, 4,000, 4,500, 5,000 or more padlock probes).

### Compositions

The present disclosure also features compositions related to determining the presence and/or location of exogenous target nucleic acids. In some embodiments, the presence and/or location of both endogenous target nucleic acids and exogenous target nucleic acids are determined with the compositions described herein.

Provided herein are compositions including: a target nucleic acid, and a plurality of padlock probes, where a padlock probe of the plurality of padlock probes includes a first sequence substantially complementary to the target nucleic acid, a capture probe binding domain complementary to a capture domain of a capture probe on a substrate, a cleavage site, and a second sequence substantially complementary to the target nucleic acid, where the padlock probe is hybridized to the target nucleic acid.

In some embodiments, the first and second sequences substantially complementary to the target nucleic acid hybridize adjacent sequences of the target nucleic acid. In some embodiments, the first and second sequences substantially complementary to the target nucleic acid hybridize to non-adjacent sequences (e.g., as described herein). In such embodiments, an extension (i.e., a gap-fill reaction) occurs to extend either the first or second sequence, followed by ligation.

Also provided herein are compositions including: a plurality of ligated padlock probes, where a ligated padlock probe of the plurality of ligated padlock probes includes a first sequence substantially complementary to a target nucleic acid, a capture probe binding domain complementary to a capture domain of a capture probe on a substrate, a cleavage site, and a second sequence substantially complementary to the target nucleic acid, where the ends of the ligated probe are ligated to each other, and where the ligated probe is hybridized to the target nucleic acid.

Also provided herein are compositions including a plurality of linear padlock probes, where a linear padlock probe of the plurality of linear probes including a first sequence substantially complementary to a target nucleic acid, a capture probe binding domain complementary to a capture domain of a capture probe on a substrate, a cleavage site, and a second sequence substantially complementary to a target nucleic acid, where the linear padlock probe is hybridized to the capture domain of the capture probe on the substrate.

In some embodiments, the substrate includes a plurality of capture probes, where the capture probe of the plurality of capture probes includes: i) a spatial barcode and ii) the capture domain. In some embodiments, the capture includes one or more functional domains.

In some embodiments, the linear padlock probe hybridized to the capture domain of the capture probe on the substrate is extended, thereby generating an extended linear padlock probe. In some embodiments, the extended linear padlock probe includes a complement of the spatial barcode and a complement of the one or more functional domains.

In some embodiments, the capture probe is extended using the hybridized linear padlock probe as a template, thereby generating an extended capture probe. The extended capture probe includes complementary sequences to those present in the linear padlock probe.

In some embodiments, the composition includes a ligase selected from a PBCV-1 DNA ligase, a Chlorella virus DNA ligase, a single stranded DNA ligase, or a T4 DNA ligase. In some embodiments, the composition includes an RNase.

In some embodiments, the composition includes a polymerase. In some embodiments, the polymerase is a DNA polymerase and/or a reverse transcriptase.

In some embodiments, the composition includes one or more restriction enzymes.

In some embodiments, the composition includes an endoribonuclease, optionally, where the endoribonuclease is one or more of RNase A, RNase C, RNase H, and RNase I.

In some embodiments, the composition includes one or more permeabilization reagents. In some embodiments, the one or more permeabilizations reagents include one or more proteases, a DNase, an RNase, a lipase, a detergent, and combinations thereof. In some embodiments, the one or more proteases include pepsin, proteinase K, collagenase, cellulase, and combinations thereof.

In some embodiments, the capture probe includes one or more functional domains, a unique molecular identifier (UMI), a cleavage domain, or combinations thereof. In some embodiments, the one or more functional domains includes a sequencing specific site or a primer binding site. In some embodiments, the capture domain includes a homopolymeric sequence. In some embodiments, where the homopolymeric sequence includes a poly(T) sequence. In some embodiments, the capture domain includes a fixed sequence (e.g., a fixed sequence as defined herein).

In some embodiments, the target nucleic acid is RNA. In some embodiments, the RNA is mRNA. In some embodiments, the target nucleic acid is endogenous to the biological sample. In some embodiments, the target nucleic acid is exogenous to the biological sample.

Non-limiting examples of RNA include various types of coding and non-coding RNA (including both endogenous and exogenous target nucleic acids). Examples of the different types of RNA target nucleic acids include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA). The RNA can be from an RNA virus, for example RNA viruses from Group III, IV or V of the Baltimore classification system. The RNA can be from a retrovirus, such as a virus from Group VI of the Baltimore classification system. The RNA can be from a fungus, for example 18S ribosomal RNA or internal transcribed spacer (ITS) region ribosomal RNA.

In some embodiments, the composition includes a biological sample (e.g., any of the biological samples described herein). In some embodiments, the biological sample is a tissue section. In some embodiments, the biological sample is a tissue sample.

### Kits

The present disclosure also features kits for determining the presence and/or location of exogenous target nucleic acids. In some embodiments, the presence and/or location of both endogenous target nucleic acids and exogenous target nucleic acids are determined with the kits described herein.

Also provided herein are kits including: a) a substrate including a plurality of capture probes, where a capture probe of the plurality of capture probes includes: i) a spatial barcode and ii) a capture domain (e.g., any of the capture domains described herein); b) a plurality of padlock probes, where a padlock probe of the plurality of padlock probes includes a first sequence substantially complementary to a target nucleic acid, a capture probe binding domain (e.g., a sequence substantially complementary to a capture domain), a cleavage site, a functional domain (as defined herein), and a second sequence substantially complementary to the target nucleic acid; and c) one or more enzymes (e.g., any of the enzymes described herein).

In some embodiments, the one or more enzymes includes a ligase. Non-limiting examples of exemplary ligases include, but are not limited to, Tth DNA ligase, Taq DNA ligase, Thermococcus sp. DNA ligase, PBCV-1 DNA Ligase, and Chlorella virus DNA Ligase. In some embodiments, the one or more enzymes includes a polymerase (e.g., a DNA polymerase). In some embodiments, the one or more enzymes includes a reverse transcriptase.

In some embodiments, the one or more enzymes includes an endoribonuclease (e.g., an RNase). In some embodiments, the RNase is one or more of RNase A, RNase C, RNase H1, RNase H2, and RNase I.

In some embodiment, the one or more enzymes includes one or more restriction enzymes (e.g., any of the restriction enzymes described herein).

In some embodiments, the kit includes one or more permeabilization reagents. In some embodiments, the one or more permeabilizations reagents include one or more proteases, a DNase, an RNase, a lipase, a detergent, and combinations thereof. In some embodiments, the one or more proteases include pepsin, proteinase K, collagenase, cellulase, and combinations thereof.

In some embodiments, the capture probe includes one or more functional domains, a cleavage domain, a unique molecular identifier, and combinations thereof. In some embodiments, the one or more functional domains include a sequencing specific site or a primer binding site.

In some embodiments, the kit includes instructions for performing any of the methods described herein.

### Library Preparation

In some embodiments, the nucleic acid products generated on the substrate (e.g., the extended capture probe, the extended linear padlock probe), and/or amplicons of such products, can be prepared for downstream applications, such as generation of a sequencing library for next-generation sequencing. Generating sequencing libraries are known in the art. For example, the extended products described herein can be collected for downstream amplification steps. The amplification products can be amplified using PCR, where primer binding sites flank the spatial barcode and products described above, or a complement thereof, generating a spatially tagged sequencing library. In some embodiments, the library preparation can be quantitated and/or quality controlled to verify the success of the library preparation steps. The library amplicons are sequenced and analyzed to decode spatial information of the nucleic acid products generated on the substrate (e.g., the extended capture probe, the extended linear padlock probe).

Alternatively, or additionally, the amplicons can be enzymatically fragmented and/or size-selected in order to provide for desired amplicon size. In some embodiments, when utilizing an Illumina® library preparation methodology, for example, P5 and P7, sequences can be added to the amplicons thereby allowing for capture of the library preparation on a sequencing flow cell (e.g., on Illumina sequencing instruments). Additionally, i7 and i5 can index sequences be added as sample indexes if multiple libraries are to be pooled and sequenced together. Further, Read 1 and Read 2 sequences can be added to the library for sequencing purposes, if not already present on the capture probe on the substrate and incorporated into the extended products. The aforementioned sequences can be added to a library preparation sample, for example, via End Repair, A-tailing, Adaptor Ligation, and/or PCR. The cDNA fragments can then be sequenced using, for example, paired-end sequencing using TruSeq Read 1 and TruSeq Read 2 as sequencing primer sites, although other methods are known in the art.

### EXAMPLES

### Example 1. Determination of the Presence and/or Location of Target Nucleic Acids in a Biological Sample via Padlock Probes

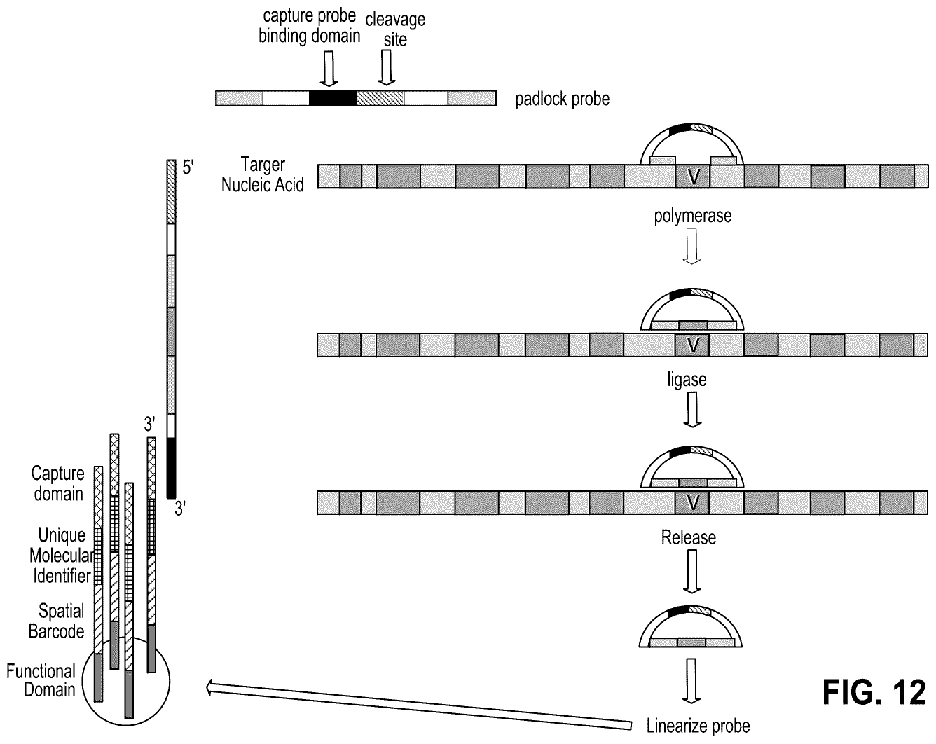

The present disclosure features padlock probes in combination with a substrate (e.g., a first substrate) including spatially barcoded capture probes to detect target nucleic acids. The methods described herein can detect both endogenous target nucleic acids in addition to exogenous target nucleic acids via the use of padlock probes. FIG. 12 shows an exemplary schematic to detect exogenous target nucleic acids via a padlock probe. Beginning at the top FIG. 12 shows an exemplary padlock probe of the present disclosure that includes a first and a second sequence that are substantially complementary to a target nucleic acid. The padlock probe also includes a capture probe binding domain (e.g., as defined herein) and a cleavage site. Optionally, the padlock probe can include one or more functional domains (e.g., as defined herein).

A plurality of padlock probes is contacted with the biological sample and FIG. 12 shows a padlock probe hybridized to the target nucleic acid (e.g., an exogenous target nucleic acid).

In some examples, the first and second sequences substantially complementary to the target nucleic acid hybridize to adjacent sequences. The hybridized first and second sequences are ligated (e.g., ligated with any of the ligases described herein), thereby generating a ligated padlock probe (e.g., as defined herein).

Alternatively, the first and second sequences hybridize to non-adjacent sequences of the target nucleic acid and either the first or the second sequence is extended (e.g., extended with a polymerase; “gap-filled”) to generate an extended padlock probe (e.g., as defined herein), followed by ligation. Either the first sequence or the second sequence is extended depending on the orientation of the padlock probe hybridized to the target nucleic acid. In some examples, the first and second sequences substantially complementary to the target nucleic acid hybridize to conserved regions of the exogenous target nucleic acid. For example, ribosomal RNA from bacteria or fungal sources includes conserved regions that are found in multiple species of bacteria or fungi, respectively. As such, padlock probes designed to hybridize to these conserved regions can detect nucleic acids from more than one species of bacteria and/or fungus. In some examples, the region between the first and second sequences of the exogenous target nucleic acid (shown as “V” in FIG. 12) includes a variable region. The sequence of the variable region can be used to determine the specific bacterial or fungal species. Although FIG. 12 shows a variable region flanked by a padlock probe, the intervening region between the first and second sequences hybridized to the target nucleic acid can be any nucleic acid sequence.

After ligation, the ligated padlock probe is released from the target nucleic and linearized. Releasing the ligated probe from the target nucleic acid can be achieved via various methods. In some examples, the ligated padlock probe is released via an endoribonuclease (e.g., an RNase e.g., any of the RNases described herein). In some examples, the ligated padlock probe is released via denaturation. In some examples, denaturation includes use of potassium hydroxide (KOH). In some examples, denaturation includes use of heat.

The ligated padlock probe is linearized via the cleavage site shown in FIG. 12. In some examples, the cleavage site is a restriction enzyme cleavage site. Any suitable restriction enzyme cleavage site and its respective restriction enzyme can be used to cleave the ligated padlock probe. The result of cleaving the padlock probe is a linearized padlock probe that includes the various domains described herein.

In some examples, releasing the ligated padlock probe from the target nucleic acid and linearizing the ligated padlock probe occur sequentially, in either order. In some examples, releasing the ligated padlock probe from the target nucleic acid and linearizing the ligated padlock probe occur simultaneously.

Next, the capture probe binding domain of the linearized padlock probe is captured (e.g., hybridizes) to the capture domain of the capture probe on the substrate (FIG. 12 bottom). The capture probe can be attached (directly or indirectly) to the substrate by its 5′ end. The 3′ end of the capture probe (e.g., the capture domain) can be extended after capture of the linearized padlock probe. The capture probe can include one or more functional domains (as defined herein), a spatial barcode, a unique molecular identifier, a cleavage domain, or combinations thereof. After capture, the capture probe can be extended using the linearized padlock probe as a template, thereby generating an extended capture probe and/or the linearized padlock probe can be extended using the capture probe as a template, thereby generating an extended linearized padlock probe (not shown). Either the extended linearized padlock probe or the extended capture probe can be removed from the substrate, optionally amplified, and prepared for downstream analysis (e.g., sequencing).

Optionally, the capture probes shown in FIG. 12 can also capture endogenous target nucleic acids. For example, capture probes with a poly(T) capture domain can capture (e.g., hybridize) to target nucleic acids with poly(A) tails, such as mRNA found within the biological sample, or proxies thereof (e.g., ligation products) or analyte capture agent-associated oligonucleotides. Thus, while not shown in FIG. 12, the methods described herein contemplate the capture and analysis (e.g., sequencing) of both endogenous target nucleic acids (i.e., mRNA), as well as exogenous target nucleic acids via padlock probes, as well as other analytes (e.g., protein).

### EMBODIMENTS

Embodiment 1 is a method for determining a location of a target nucleic acid in a biological sample comprising: a) providing a first substrate comprising a plurality of capture probes, wherein a capture probe of the plurality of capture probes comprises: i) a spatial barcode and ii) a capture domain; b) contacting the biological sample with a plurality of padlock probes, wherein a padlock probe of the plurality of padlock probes comprises a first sequence substantially complementary to the target nucleic acid, a capture probe binding domain, a cleavage site, and a second sequence substantially complementary to the target nucleic acid; c) hybridizing the padlock probe to the target nucleic acid; d) extending the second sequence substantially complementary to the target nucleic acid, thereby generating an extended padlock probe; e) ligating the ends of the extended padlock probe to each other, thereby generating a ligated padlock probe; f) cleaving the cleavage site of the ligated padlock probe, thereby generating a linear padlock probe; g) hybridizing the capture probe binding domain of the linear padlock probe to the capture domain of the capture probe on the first substrate; and h) determining the sequence of (i) the spatial barcode, or a complement thereof, and (ii) all or a part of the sequence of the linear padlock probe, or a complement thereof; and using the sequences of (i) and (ii) to determine the location of the target nucleic acid in the biological sample.

Embodiment 2 is the method of embodiment 1, wherein the biological sample is disposed on the first substrate comprising the plurality of capture probes.

Embodiment 3 is the method of embodiment 1, wherein the biological sample is disposed on a second substrate.

Embodiment 4 is the method of embodiment 3, wherein the method further comprises aligning the second substrate comprising the biological sample with the first substrate, such that at least a portion of the biological sample is aligned with at least a portion of the first substrate.

Embodiment 5 is the method of any one of embodiments 1-4, wherein the first substrate comprises one or more features.

Embodiment 6 is the method of embodiment 5, wherein the one or more features are selected from the group consisting of: a spot (e.g., an inkjet spot, a masked spot), a pit, a post, a well, a ridge, a hydrogel pad, and a bead.

Embodiment 7 is he method of any one of embodiments 1-6, wherein the capture probe further comprises a cleavage domain and/or one or more functional domains.

Embodiment 8 is the method of embodiment 7, wherein the capture probe further comprises a unique molecular identifier.

Embodiment 9 is the method of embodiment 7, wherein the one or more functional domains comprises a sequencing specific site or a primer binding site.

Embodiment 10 is the method of any one of embodiments 1-9, wherein the hybridizing in step (c) comprises hybridizing the first sequence substantially complementary to the target nucleic acid and the second sequence substantially complementary to the target nucleic acid of the padlock probe to the target nucleic acid.

Embodiment 11 is the method of embodiment 10, wherein the first sequence substantially complementary to the target nucleic acid and the second sequence substantially complementary to the target nucleic acid hybridize to conserved regions of the target nucleic acid.

Embodiment 12 is the method of embodiment 10 or 11, wherein the first sequence substantially complementary to the target nucleic acid and the second sequence substantially complementary to the target nucleic acid flank a variable region of the target nucleic acid.

Embodiment 13 is the method of any one of embodiments 1-12, wherein extending the second sequence substantially complementary to the target nucleic acid comprises a gap-fill reaction.

Embodiment 14 is the method of any one of embodiments 1-13, wherein hybridizing the padlock probe to the target nucleic acid and extending the second sequence substantially complementary to the target nucleic acid occur sequentially.

Embodiment 15 is the method of any one of embodiments 1-13, wherein hybridizing the padlock probe to the target nucleic acid and extending the second sequence substantially complementary to the target nucleic acid occur simultaneously.

Embodiment 16 is the method of any one of embodiments 1-15, wherein ligating the ends of the extended padlock probe comprises ligating a first end of the extended padlock probe to a second end of the extended padlock probe.

Embodiment 17 is the method of any one of embodiments 1-16, wherein the padlock probe comprises in a 5′ to 3′ direction: (i) the first sequence substantially complementary to the target nucleic acid; (ii) the capture probe binding domain; (iii) the cleavage site; and (iv) the second sequence substantially complementary to the target nucleic acid.

Embodiment 18 is the method of embodiment 17, wherein the padlock probe further comprises a functional domain.

Embodiment 19 is the method of embodiment 18, wherein the functional domain is a sequencing specific site or a primer binding site.

Embodiment 20 is the method of any one of embodiments 17-19, wherein the functional domain is disposed 3′ to the cleavage site, 5′ to the second sequence substantially complementary to the target nucleic acid, or a combination thereof.

Embodiment 21 is the method of any one of embodiments 1-13, further comprising releasing the ligated padlock probe from the target nucleic acid, optionally wherein the releasing is performed prior to step (g).

Embodiment 22 is the method of embodiment 21, wherein the releasing comprises use of one or more RNases.

Embodiment 23 is the method of embodiment 22, wherein the one or more RNases comprises RNase A, RNase C, RNase H, or RNase I.

Embodiment 24 is the method of embodiment 23, wherein the RNase H comprises one or both of RNase H1 and RNase H2.

Embodiment 25 is the method of embodiment 21, wherein the releasing comprises the use of KOH.

Embodiment 26 is the method of embodiment 21, wherein the releasing comprises the use of heat.

Embodiment 27 is the method of any one of embodiments 1-26, wherein extending the second sequence substantially complementary to the target nucleic acid comprises use of a polymerase.

Embodiment 28 is the method of embodiment 27, wherein the polymerase is a DNA polymerase.

Embodiment 29 is the method of embodiment 27, wherein the polymerase is a reverse transcriptase.

Embodiment 30 is the method of any one of embodiments 1-29, wherein the padlock probe comprises about 200 nucleotides to about 500 nucleotides.

Embodiment 31 is the method of embodiment 30, wherein the padlock probe comprises about 250 nucleotides to about 450 nucleotides.

Embodiment 32 is the method of embodiment 30 or 31, wherein the padlock probe comprises about 300 nucleotides.

Embodiment 33 is the method of any one of embodiments 1-32, wherein ligating the ends of the extended padlock probe is performed using a ligase selected from the group consisting of: Tth DNA ligase, Taq DNA ligase, Thermococcus sp. DNA ligase, PBCV-1 DNA Ligase, and Chlorella virus DNA Ligase.

Embodiment 34 is the method of any one of embodiments 1-33, wherein the cleavage site is a restriction enzyme site.

Embodiment 35 is the method of embodiment 34, wherein the restriction enzyme site is cleaved by one or more restriction enzymes.

Embodiment 36 is the method of any one of embodiments 1-35, wherein the capture domain is a homopolymeric sequence.

Embodiment 37 is the method of embodiment 36, wherein the homopolymeric sequence is a poly(T) sequence.

Embodiment 38 is the method of any one of embodiments 1-35, wherein the capture domain is a fixed sequence.

Embodiment 39 is the method of any one of embodiments 1-38, further comprising extending the capture probe using the linear padlock probe as a template, thereby generating an extended capture probe.

Embodiment 40 is the method of any one of embodiments 1-39, further comprising extending the linear padlock probe using the capture probe as a template, thereby generating an extended linear padlock probe.

Embodiment 41 is the method of any one of embodiments 1-40, wherein the determining step comprises sequencing.

Embodiment 42 is the method of embodiment 41, wherein the sequencing comprises high-throughput sequencing.

Embodiment 43 is the method of any one of embodiments 1-40, wherein the determining step comprises fluorescent hybridization.

Embodiment 44 is the method of any one of embodiments 1-43, wherein the biological sample is derived from a mammal, a plant, a nematode, or a fungus.

Embodiment 45 is the method of any one of embodiments 1-44, wherein the target nucleic acid is endogenous to the biological sample.

Embodiment 46 is the method of embodiment 45, wherein the target nucleic acid is RNA.

Embodiment 47 is the method of embodiment 46, wherein the RNA is mRNA.

Embodiment 48 is the method of any one of embodiments 1-44, wherein the target nucleic acid is exogenous to the biological sample.

Embodiment 49 is the method of embodiment 48, wherein the target nucleic acid is archaeal RNA, bacterial RNA, fungal RNA, or a combination thereof.

Embodiment 50 is the method of embodiment 49, wherein the bacterial RNA is bacterial ribosomal RNA.

Embodiment 51 is the method of embodiment 51, wherein the bacterial ribosomal RNA is 16S ribosomal RNA or 5S ribosomal RNA.

Embodiment 52 is the method of embodiment 49, wherein the fungal RNA is 18S ribosomal RNA or internal transcribed spacer (ITS) region ribosomal RNA.

Embodiment 53 is the method of any one of embodiments 1-52, wherein the target nucleic acid is exogenous to the biological sample, and wherein the method further comprises hybridizing a second target nucleic acid, or a proxy thereof, to a second capture domain of a second capture probe comprised in the plurality of capture probes, wherein the second target nucleic acid is endogenous to the biological sample.

Embodiment 54 is the method of embodiment 53, further comprising determining the location of the endogenous second target nucleic acid and the exogenous target nucleic acid in the biological sample, optionally wherein the method further comprises determining the sequence of (iii) a second spatial barcode comprised in the second capture probe, or a complement thereof, and (iv) all or a part of the sequence of the endogenous second target nucleic acid or a proxy thereof, or a complement thereof; and using the sequences of (iii) and (iv) to determine the location of the endogenous second target nucleic acid in the biological sample.

Embodiment 55 is the method of embodiment 54, wherein the determining comprises sequencing.

Embodiment 56 is the method of any one of embodiments 1-55, further comprising imaging the biological sample.

Embodiment 57 is the method of any one of embodiments 1-56, further comprising staining the biological sample.

Embodiment 58 is the method of embodiment 57, wherein the staining comprises use of eosin and/or hematoxylin.

Embodiment 59 is the method of embodiment 57, wherein the staining comprises the use of a detectable label selected from the group consisting of a radioisotope, a fluorophore, a chemiluminescent compound, a bioluminescent compound, or a combination thereof.

Embodiment 60 is the method of any one of embodiments 1-59, further comprising migrating the linear padlock probe from the biological sample to the first substrate, optionally wherein the migrating comprises electrophoresis.

Embodiment 61 is the method of any one of embodiments 1-60, wherein the method further comprises permeabilizing the biological sample.

Embodiment 62 is the method of embodiment 61, wherein the permeabilizing comprises use of a permeabilization reagent selected from Proteinase K, pepsin, and/or collagenase.

Embodiment 63 is the method of any one of embodiments 1-62, wherein cleaving the cleavage site of the ligated padlock probe, releasing the ligated padlock probe from the target nucleic acid, and permeabilizing the biological sample are performed simultaneously.

Embodiment 64 is the method of any one of embodiments 1-63, wherein the biological sample is a tissue sample.

Embodiment 65 is the method of embodiment 64, wherein the tissue sample is a fixed tissue sample.

Embodiment 66 is the method of embodiment 65, wherein the fixed tissue sample is a methanol-fixed tissue sample, an acetone-fixed tissue sample, a paraformaldehyde-fixed tissue sample, or a formalin-fixed paraffin-embedded tissue sample.

Embodiment 67 is the method of embodiment 64, wherein the tissue sample is a fresh-frozen tissue sample.

Embodiment 68 is the method of any one of embodiments 1-64, wherein the biological sample is a tissue section.

Embodiment 69 is the method of embodiment 68, wherein the tissue section is a fixed tissue section.

Embodiment 70 is the method of embodiment 69, wherein the fixed tissue section is a methanol-fixed tissue section, an acetone-fixed tissue section, a paraformaldehyde-fixed tissue section, or a formalin-fixed paraffin-embedded tissue section.

Embodiment 71 is the method of embodiment 57, wherein the tissue section is a fresh-frozen tissue section.

Embodiment 72 is a kit comprising: a substrate comprising a plurality of capture probes, wherein a capture probe of the plurality of capture probes comprises: i) a spatial barcode and ii) a capture domain; a plurality of padlock probes, wherein a padlock probe of the plurality of padlock probes comprises a first sequence substantially complementary to a target nucleic acid, a capture probe binding domain, a cleavage site, a functional domain, and a second sequence substantially complementary to the target nucleic acid; and one or more enzymes.

Embodiment 73 is the kit of embodiment 72, wherein the one or more enzymes comprises a ligase, a polymerase, a reverse transcriptase, an endoribonuclease, one or more restriction enzymes, and combinations thereof.

Embodiment 74 is the kit of embodiment 72 or 73, wherein the kit further comprises one or more permeabilization reagents.

Embodiment 75 is the kit of embodiment 74, wherein the one or more permeabilizations reagents include one or more proteases, a DNase, an RNase, a lipase, a detergent, and combinations thereof.

Embodiment 76 is the kit of embodiment 75, wherein the one or more proteases comprise pepsin, proteinase K, collagenase, cellulase, and combinations thereof.

Embodiment 77 is the kit of any one of embodiments 72-76, wherein the capture probe further comprises one or more functional domains, a cleavage domain, a unique molecular identifier, and combinations thereof, optionally wherein the one or more functional domains comprise a sequencing specific site or a primer binding site.

Embodiment 78 is the kit of any one of embodiments 72-78, wherein the padlock probe includes in a 5′ to 3′ direction: (i) the first sequence substantially complementary to the target nucleic acid; (ii) the capture probe binding domain; (iii) the cleavage site; and (iv) the second sequence substantially complementary to the target nucleic acid, optionally wherein the target nucleic acid is selected from archaeal RNA, bacterial RNA, fungal RNA, or a combination thereof.

Embodiment 79 is the kit of any one of embodiments 72-78, further comprising instructions for performing the method of any one of embodiments 1-71.

Embodiment 80 is a composition comprising: a target nucleic acid, and a plurality of padlock probes, wherein a padlock probe of the plurality of padlock probes comprises a first sequence substantially complementary to the target nucleic acid, a capture probe binding domain complementary to a capture domain of a capture probe on a substrate, a cleavage site, and a second sequence substantially complementary to the target nucleic acid, wherein the padlock probe is hybridized to the target nucleic acid.

Embodiment 81 is a composition comprising: plurality of ligated padlock probes, wherein a ligated padlock probe of the plurality of ligated padlock probes comprises a first sequence substantially complementary to a target nucleic acid, a capture probe binding domain complementary to a capture domain of a capture probe on a substrate, a cleavage site, and a second sequence substantially complementary to the target nucleic acid, wherein the ends of the ligated probe are ligated to each other, and wherein the ligated probe is hybridized to the target nucleic acid.

Embodiment 82 is the composition of embodiment 80 or 81, the substrate comprises a plurality of capture probes, wherein the capture probe of the plurality of capture probes comprises: i) a spatial barcode and ii) the capture domain.

Embodiment 83 is a composition comprising: a plurality of linear padlock probes, wherein a linear padlock probe of the plurality of linear probes comprising a first sequence substantially complementary to a target nucleic acid, a capture probe binding domain complementary to a capture domain of a capture probe on a substrate, a cleavage site, and a second sequence substantially complementary to a target nucleic acid, wherein the linear padlock probe is hybridized to the capture domain of the capture probe on the substrate.

Embodiment 84 is the composition of embodiment 83, wherein the capture probe further comprises one or more functional domains and a spatial barcode.

Embodiment 85 is the composition of embodiment 83 or 84, wherein the linear padlock probe hybridized to the capture domain of the capture probe on the substrate is extended, thereby generating an extended linear padlock probe.

Embodiment 86 is the composition of embodiment 85, wherein the extended linear padlock probe comprises a complement of the spatial barcode and a complement of the one or more functional domains.

Embodiment 87 is the composition of any one of embodiments 80-86, further comprising a ligase.

Embodiment 88 is the composition of any one of embodiments 80-87, further comprising a polymerase.

Embodiment 89 is the composition of embodiment 88, wherein the polymerase is a DNA polymerase and/or a reverse transcriptase.

Embodiment 90 is the composition of any one of embodiments 80-89, further comprising one or more restriction enzymes.

Embodiment 91 is the composition of any one of embodiments 80-90, further comprising an endoribonuclease, optionally, wherein the endoribonuclease is one or more of RNase A, RNase C, RNase H, and RNase I.

Embodiment 92 is the composition of any one of embodiments 80-91, further comprising one or more permeabilization reagents.

Embodiment 93 is the composition of embodiment 92, wherein the one or more permeabilizations reagents include one or more proteases, a DNase, an RNase, a lipase, a detergent, and combinations thereof.

Embodiment 94 is the composition of embodiment 93, wherein the one or more proteases comprise pepsin, proteinase K, collagenase, cellulase, and combinations thereof.

Embodiment 95 is the composition of any one of embodiments 80-94, wherein the capture probe further comprises one or more functional domains, a cleavage domain, a unique molecular identifier, and combinations thereof.

Embodiment 96 is the composition of embodiment 95, wherein the one or more functional domains comprise a sequencing specific site or a primer binding site.

Embodiment 97 is a method for determining a presence and/or location of a microbial target nucleic acid in a biological sample comprising: providing a substrate comprising a plurality of capture probes, wherein a capture probe of the plurality of capture probes comprises: i) a spatial barcode and ii) a capture domain; contacting the biological sample with a plurality of padlock probes, wherein a padlock probe of the plurality of padlock probes comprises a first sequence substantially complementary to the microbial target nucleic acid, a capture probe binding domain, a cleavage site, and a second sequence substantially complementary to the microbial target nucleic acid; hybridizing the padlock probe to the microbial target nucleic acid; ligating ends of the extended padlock probe to each other, thereby generating a ligated padlock probe; cleaving the cleavage site of the ligated padlock probe, thereby generating a linear padlock probe; hybridizing the capture probe binding domain of the linear padlock probe to the capture domain of the capture probe on the substrate; and determining the sequence of (i) the spatial barcode and (ii) all or a part of the sequence of the linear padlock probe; and using the sequences of (i) and (ii) to determine the presence and/or location of the microbial target nucleic acid in the biological sample.

Embodiment 98 is a method for determining a location of a target nucleic acid in a biological sample comprising: providing a substrate comprising a plurality of capture probes, wherein a capture probe of the plurality of capture probes comprises: i) a spatial barcode and ii) a capture domain; contacting the biological sample with a plurality of padlock probes, wherein a padlock probe of the plurality of padlock probes comprises a first sequence substantially complementary to the target nucleic acid, a capture probe binding domain, a cleavage site, and a second sequence substantially complementary to the target nucleic acid; hybridizing the padlock probe to the target nucleic acid; ligating ends of the padlock probe to each other, thereby generating a ligated padlock probe; cleaving the cleavage site of the ligated padlock probe, thereby generating a linear padlock probe; hybridizing the capture probe binding domain of the linear padlock probe to the capture domain of the capture probe on the substrate; and determining the sequence of (i) the spatial barcode, or a complement thereof, and (ii) all or a part of the sequence of the linear padlock probe, or a complement thereof; and using the sequences of (i) and (ii) to determine the location of the target nucleic acid in the biological sample.

Embodiment 99 is the method of embodiment 53 or 54, wherein the proxy of the second target nucleic acid is a ligation product.

Embodiment 100 is the method of embodiment 99, wherein the ligation product is generated by hybridizing a first and second oligonucleotide to a first and second sequence of the second target nucleic acid, respectively, and ligating the first and second oligonucleotides to each other, thereby generating the ligation product.

Embodiment 101 is the method of any one of embodiments 1-69 or 97-100, for use in determining a presence and/or location of a microbe in a biological sample, optionally wherein the microbe is selected from fungi, bacteria, archaea, or a combination thereof, optionally wherein the microbe is a pathogenic microbe.

What is claimed is:

1. A method for determining a location of a target nucleic acid in a biological sample comprising:
a) contacting the biological sample with a plurality of padlock probes, wherein a padlock probe of the plurality of padlock probes comprises a first sequence substantially complementary to the target nucleic acid, a capture probe binding domain, a cleavage site, and a second sequence substantially complementary to the target nucleic acid;
b) hybridizing the padlock probe to the target nucleic acid;
c) extending the second sequence substantially complementary to the target nucleic acid, thereby generating an extended padlock probe;
d) ligating a first end of the extended padlock probe to a second end of the extended padlock probe, thereby generating a ligated padlock probe;
e) cleaving the cleavage site of the ligated padlock probe, thereby generating a linear padlock probe;
f) hybridizing the capture probe binding domain of the linear padlock probe to a capture domain of a capture probe on a first substrate, wherein the capture probe is comprised in a plurality of capture probes on the first substrate, the capture probe comprising: i) a spatial barcode and ii) the capture domain; and
g) determining the sequence of (i) the spatial barcode, or a complement thereof, and (ii) all or a part of the sequence of the linear padlock probe, or a complement thereof; and using the sequences of (i) and (ii) to determine the location of the target nucleic acid in the biological sample.

2. The method of claim 1, wherein the biological sample is disposed on the first substrate comprising the plurality of capture probes.

3. The method of claim 1, wherein the biological sample is disposed on a second substrate.

4. The method of claim 3, wherein the method further comprises aligning the second substrate comprising the biological sample with the first substrate, such that at least a portion of the biological sample is aligned with at least a portion of the first substrate.

5. The method of claim 1, wherein the capture probe further comprises a unique molecular identifier, a cleavage domain, a sequencing specific site, and/or a primer binding site.

6. The method of claim 1, wherein the first sequence substantially complementary to the target nucleic acid and the second sequence substantially complementary to the target nucleic acid hybridize to conserved regions of the target nucleic acid.

7. The method of claim 1, wherein the first sequence substantially complementary to the target nucleic acid and the second sequence substantially complementary to the target nucleic acid flank a variable region of the target nucleic acid.

8. The method of claim 1, wherein the padlock probe comprises in a 5′ to 3′ direction: (i) the first sequence substantially complementary to the target nucleic acid; (ii) the capture probe binding domain; (iii) the cleavage site; and (iv) the second sequence substantially complementary to the target nucleic acid.

9. The method of claim 8, wherein the padlock probe further comprises a functional domain, optionally a sequencing specific site or a primer binding site.

10. The method of claim 1, further comprising releasing the ligated padlock probe from the target nucleic acid, optionally wherein the releasing is performed prior to step (f), wherein the releasing comprises use of one or more RNases.

11. The method of claim 1, wherein the ligating is performed using a ligase selected from the group consisting of: Tth DNA ligase, Taq DNA ligase, Thermococcus sp. DNA ligase, PBCV-1 DNA Ligase, and Chlorella virus DNA Ligase.

12. The method of claim 1, further comprising extending the capture probe using the linear padlock probe as a template, thereby generating an extended capture probe, and/or extending the linear padlock probe using the capture probe as a template, thereby generating an extended linear padlock probe.

13. The method of claim 12, wherein the determining step comprises sequencing the extended capture probe or a complement thereof, or the extended linear padlock probe or a complement thereof.

14. The method of claim 1, wherein the biological sample is derived from a mammal or a plant, optionally wherein the target nucleic acid is exogenous to the biological sample.

15. The method of claim 14, wherein the target nucleic acid comprises archaeal RNA, bacterial RNA, fungal RNA, or a combination thereof.

16. The method of claim 15, wherein the bacterial RNA is bacterial ribosomal RNA comprising 16S ribosomal RNA or 5S ribosomal RNA, or wherein the fungal RNA is fungal RNA comprising 18S ribosomal RNA or internal transcribed spacer (ITS) region ribosomal RNA.

17. The method of claim 1, wherein the biological sample is a tissue section, optionally a fixed tissue section or a fresh-frozen tissue section.

18. A kit comprising:
a) a substrate comprising a plurality of capture probes, wherein a capture probe of the plurality of capture probes comprises: i) a spatial barcode and ii) a capture domain;
b) a plurality of padlock probes, wherein a padlock probe of the plurality of padlock probes comprises a first sequence substantially complementary to a target nucleic acid, a capture probe binding domain, a cleavage site, a functional domain, and a second sequence substantially complementary to the target nucleic acid; and
c) one or more enzymes.

19. A composition comprising:
a target nucleic acid, and
a plurality of ligated padlock probes, wherein a ligated padlock probe of the plurality of ligated padlock probes comprises a first sequence substantially complementary to a target nucleic acid, a capture probe binding domain complementary to a capture domain of a capture probe on a substrate, a cleavage site, and a second sequence substantially complementary to the target nucleic acid, wherein the ends of the ligated padlock probe are ligated to each other, and wherein the ligated padlock probe is hybridized to the target nucleic acid.

20. A method for determining a presence and/or location of a microbial target nucleic acid in a biological sample comprising:
a) providing a first substrate comprising a plurality of capture probes, wherein a capture probe of the plurality of capture probes comprises: i) a spatial barcode and ii) a capture domain;
b) contacting the biological sample with a plurality of padlock probes, wherein a padlock probe of the plurality of padlock probes comprises a first sequence substantially complementary to the microbial target nucleic acid, a capture probe binding domain, a cleavage site, and a second sequence substantially complementary to the microbial target nucleic acid;
c) hybridizing the padlock probe to the microbial target nucleic acid;
d) ligating ends of the extended padlock probe to each other, thereby generating a ligated padlock probe;
e) cleaving the cleavage site of the ligated padlock probe, thereby generating a linear padlock probe;
f) hybridizing the capture probe binding domain of the linear padlock probe to the capture domain of the capture probe on the first substrate; and
g) determining the sequence of (i) the spatial barcode and (ii) all or a part of the sequence of the linear padlock probe; and using the sequences of (i) and (ii) to determine the presence and/or location of the microbial target nucleic acid in the biological sample; optionally wherein the microbial target nucleic acid is derived from a microbe selected from the group comprising fungi, bacteria, archaea, or a combination thereof, optionally wherein the microbe is a pathogenic microbe.
